SEQUENCE LISTING (10 sequences)

SEQ ID NO: 1 - DNA, 21 bp, Homo sapiens
  source 1..21: mol_type = other DNA
tgaagttgaaaaggccatcag

SEQ ID NO: 2 - DNA, 20 bp, Homo sapiens
  source 1..20: mol_type = other DNA
gaggtccaagtgttggctgt

SEQ ID NO: 3 - DNA, 22 bp, Homo sapiens
  source 1..22: mol_type = other DNA
gagaaagggaagacctcagaag

SEQ ID NO: 4 - DNA, 22 bp, Homo sapiens
  source 1..22: mol_type = other DNA
ggtgtgatcaggttgtcataga

SEQ ID NO: 5 - DNA, 20 bp, Homo sapiens
  source 1..20: mol_type = other DNA
cctgcaccaccaactgctta

SEQ ID NO: 6 - DNA, 21 bp, Homo sapiens
  source 1..21: mol_type = other DNA
ggccatccacagtcttctgag

SEQ ID NO: 7 - DNA, 20 bp, SARS-CoV-2
  source 1..20: mol_type = other DNA
gaccccaaaatcagcgaaat

SEQ ID NO: 8 - DNA, 24 bp, SARS-CoV-2
  source 1..24: mol_type = other DNA
tctggttactgccagttgaatctg

SEQ ID NO: 9 - RNA, 22 nt, SARS-CoV-2
  source 1..22: mol_type = other RNA
gtaacaaaccaaccaactttcg

SEQ ID NO: 10 - RNA, 23 nt, SARS-CoV-2
  source 1..23: mol_type = other RNA
cattgttcactgtacactcgatc

(19) United States
(12) Patent Application Publication
(10) Pub. No.: US 2025/0268848 A1
(43) Pub. Date: Aug. 28, 2025

(54) COMPOSITIONS COMPRISING ENDOSIDIN 2 FOR REDUCING SARS-COV-2 INFECTION

(71) Applicant: HENRY FORD HEALTH SYSTEM, DETROIT, MI (US)

(72) Inventors: Qing-Sheng MI, Bloomfield Hills, MI (US); Li Zhou, Bloomfield Hills, MI (US); Yi YAO, Canton, MI (US)

(21) Appl. No.: 18/574,776

(22) PCT Filed: Jul. 2, 2022
(86) PCT No.: PCT/US2022/073403

§ 371 Date: Dec. 28, 2023

Related U.S. Application Data
(60) Provisional application No. 63/217,976, filed on Jul. 2, 2021.

(51) Int. Cl. A61K 31/166; C12Q 1/37

(52) U.S. Cl. CPC A61K 31/166; C12Q 1/37; G01N 2333/948; G01N 2800/52

(57) ABSTRACT

A composition comprising ES2 prevents ACE2 translocation to the cell surface and susceptibility to SARS-CoV-2 infection. Treating a subject with a composition comprising the compound ES2 may reduce the risk of severe COVID-19. The prognosis of COVID-19 in a subject can be predicted by measuring ACE2 and inflammatory and immunosuppressive phenotypes on isolated peripheral blood mononuclear cells by flow cytometry.

### CROSS REFERENCE TO RELATED APPLICATIONS

This application claims the benefit of priority to U.S. Provisional Patent Application Ser. No. 63/217,976, filed Jul. 2, 2021 the entire content of which is incorporated herein by reference.

### REFERENCE TO THE SEQUENCE LISTING

The instant application contains a Sequence Listing which has been submitted electronically in XML format and which is hereby incorporated by reference in its entirety. Said XML copy, created on Aug. 14, 2024, is named 25824-513566_Sequence-Listing-Replacement_08-14-2024 and is 10 kilobytes in size.

### BACKGROUND

An angiotensin-converting enzyme 2 (ACE2) receptor is required for SARS-CoV-2 to enter human cells. However, emerging evidence shows SARS-CoV-2 infected lung monocytes/macrophages from COVID-19 patients barely express ACE2 mRNA, raising a question of how SARS-CoV-2 penetrates macrophages. It's also unclear whether the peripheral blood immune cells can be infected by SARS-CoV-2 and thus facilitate viral spread from circulation to other organs besides lung.

### SUMMARY

Whether human peripheral blood cells are infected by SARS-CoV-2 has been debated because immune cells lack mRNA expression of both angiotensin-converting enzyme 2 (ACE2) and transmembrane serine protease type 2 (TMPRSS2). The present disclosure demonstrates that resting primary monocytes harbor abundant cytoplasmic ACE2 and TMPRSS2 protein and that circulating exosomes contain significant ACE2 protein. In ex vivo TLR4/7/8 stimulation, cytoplasmic ACE2 quickly translocated to the monocyte cell surface independently of ACE2 transcription, while TMPRSS2 surface translocation occurred in conjunction with elevated mRNA expression. Rapid translocation of ACE2 to the monocyte cell surface was blocked by a compound that inhibits endosomal trafficking, endosidin 2 (ES2). Thus, a therapeutically effective dose of the endosomal trafficking inhibitor ES2 could prevent ACE2-mediated SARS-CoV-2 infection of immune cells.

In some embodiments, the present disclosure describes a composition comprising ES2, or a pharmaceutically acceptable salt thereof, for use in inhibiting exocytosis in an immune cell. Inhibiting exocytosis in an immune cell can reduce ACE2 translocation to the cell membrane of an immune cell. In some aspects, the present disclosure provides a composition comprising ES2, or a pharmaceutically acceptable salt thereof, for use in inhibiting endocytosis recycling. Inhibiting endocytosis recycling in an immune cell can also reduce ACE2 translocation to the cell membrane of the immune cell. SAR-CoV-2 uses the ACE2 receptor to enter cells, so reduced ACE2 expression at the immune cell surface can reduce susceptibility of the immune cell to SARS-CoV-2 infection.

In other aspects, the present disclosure describes a composition comprising ES2, or a pharmaceutically acceptable salt thereof, and a pharmaceutically acceptable excipient. In other aspects, the present disclosure provides a composition comprising ES2, wherein the composition is carried in or by a drug delivery particle. In some aspects, the present disclosure describes a drug delivery particle with an immune-cell-targeting-moiety on the surface. The immune cell targeting moiety may target receptors on immune cells or may be a ligand known to be phagocytosed by immune cells. In some aspects, the present disclosure describes a drug delivery particle carrying a composition comprising ES2, wherein a moiety on the drug delivery particle targets a macrophage or monocyte and is phagocytosed.

In some aspects, the present disclosure describes a method of reducing SARS-CoV-2 infection in a subject by administration of a therapeutically effective dosage of a composition comprising ES2, as disclosed herein, to a subject. In some aspects, the subject is a human. In some aspects, the present disclosure describes methods for administering a composition comprising ES2 enterally or parenterally. In some aspects, the present disclosure provides methods of treating a subject, wherein any of the compositions are administered by oral ingestion, inhalation, infusion (intravenous, subcutaneous, intracranial, epidural, or intramuscular), or combinations thereof.

In some aspects, the present disclosure describes methods for administering a composition comprising ES2 in a single bolus dosage. In some aspects, the present disclosure describes methods for administering a composition comprising ES2 in a repeated dosing regimen.

Some embodiments described herein are directed toward a method for determining COVID-19 prognosis in a subject, which may involve isolating PBMCs from the subject, and measuring the level of ACE2 cell surface expression by flow cytometry. The subject may be human. In some embodiments, the level of ACE2 on PBMCs is measured along with proinflammatory and immunosuppressive cell phenotypes.

In further aspects, the present disclosure describes methods for measuring proinflammatory and immunosuppressive cell phenotypes in addition to ACE2 by detecting cell surface markers informing on these phenotypes. A sample of cell surface markers comprise PD-L1, CCLS, CD11b, CD38, CD163, CD86, HLA-DR, or combinations thereof, and can be measured by flow cytometry, for example.

Some embodiments are directed to methods for measuring ACE2 and proinflammatory and immunosuppressive cell phenotypes in specific immune cell types to obtain a prognosis of COVID-19. In some aspects, the disclosure describes the isolation and sorting of PBMCS into BDCA3+ cDCs, CD16+ classic monocytes, CD68+ macrophages, or a combinations thereof, by flow cytometry and measuring ACE2 and proinflammatory and immunosuppressive cell phenotypes of specific immune cell types.

In some aspects, the disclosure describes a method wherein proinflammatory and immunosuppressive phenotypes are measured by detecting cell surface markers comprising PD-L1, CCLS, CD11 b, CD38, CD163, CD86, HLA-DR, or combination thereof by flow cytometry.

In addition, the present disclosure describes a method wherein the level of ACE2, and proinflammatory and immunosuppressive phenotypes are measured at different time points to inform on the disease prognosis of a subject with COVID-19.

### BRIEF DESCRIPTION OF FIGURES

The following figures are provided by way of example and are not intended to limit the scope of the invention.

FIG. 1A shows a flow cytometry gating strategy of PBMC major populations. Gate strategy of B cells, T cells, co14+ monocytes (MO), cDCs and pDCs in PBMCs freshly isolated from healthy subjects.

FIG. 1B shows an overlaid scatterplot of each PBMC subset represented in FIG. 1A.

FIG. 1C represents a flow cytometry analysis of ACE2 protein expression (red line) in T cells, B cells, CD14+MO, cDCs, and pDCs from fresh PBMCs isolated from healthy donors. Mouse IgG1 antibody (gray tinted) was used as isotype control.

FIG. 10 shows the frequency of cells expressing cell surface and cytoplasmic ACE2 as in FIG. 1C (n=4 biologically independent samples/group).

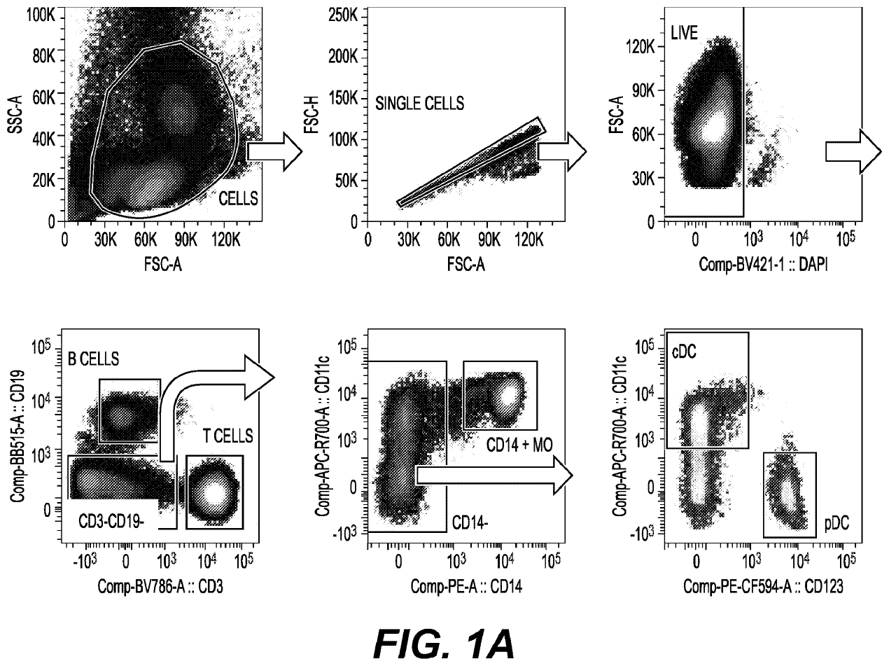

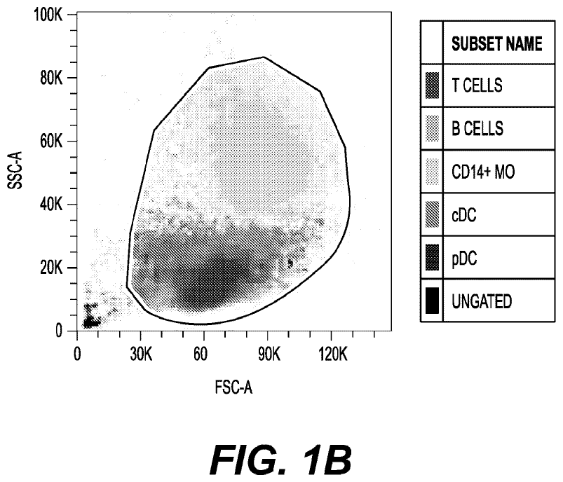

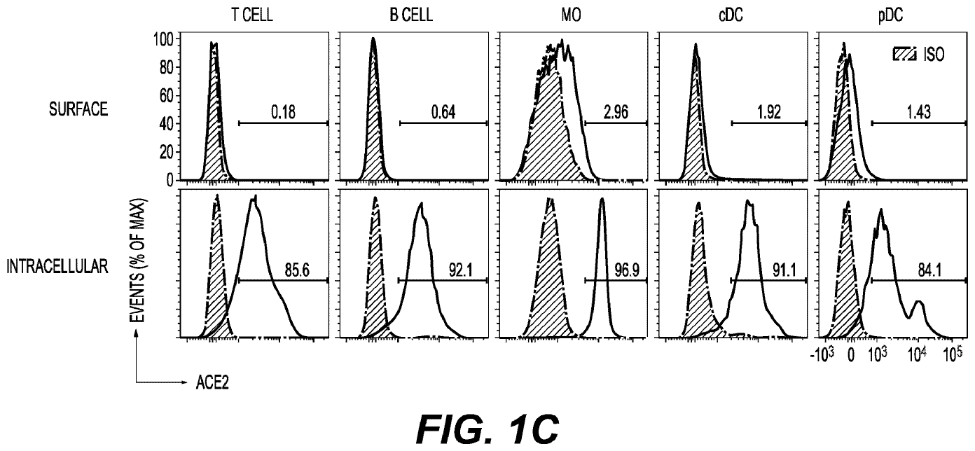

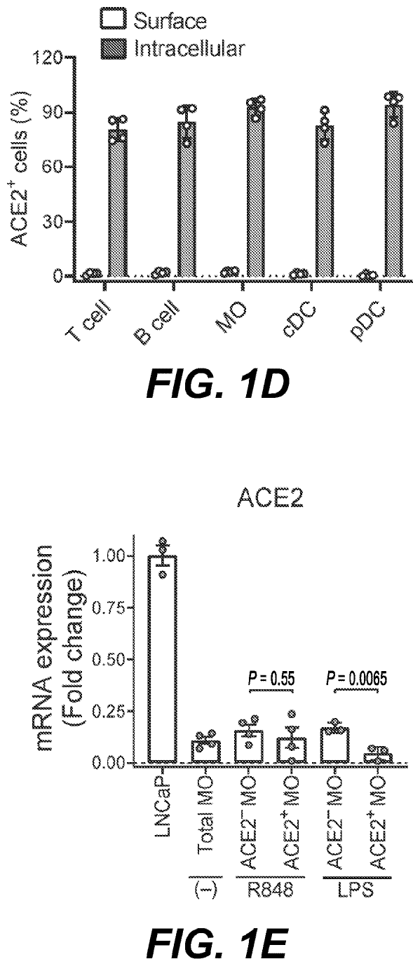

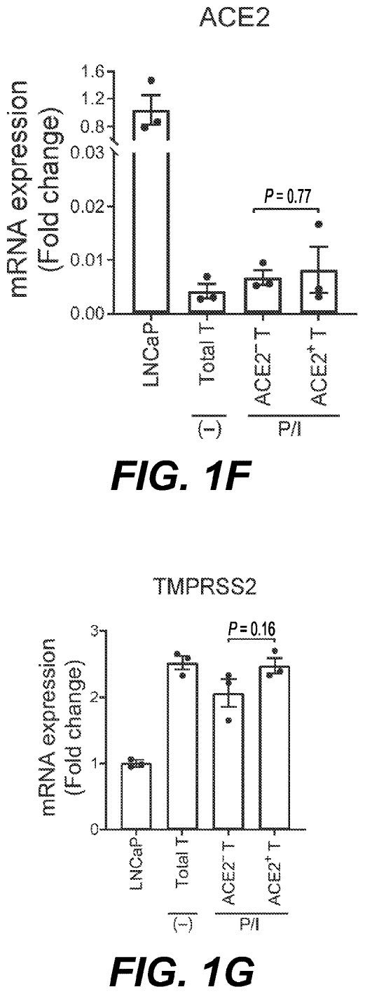

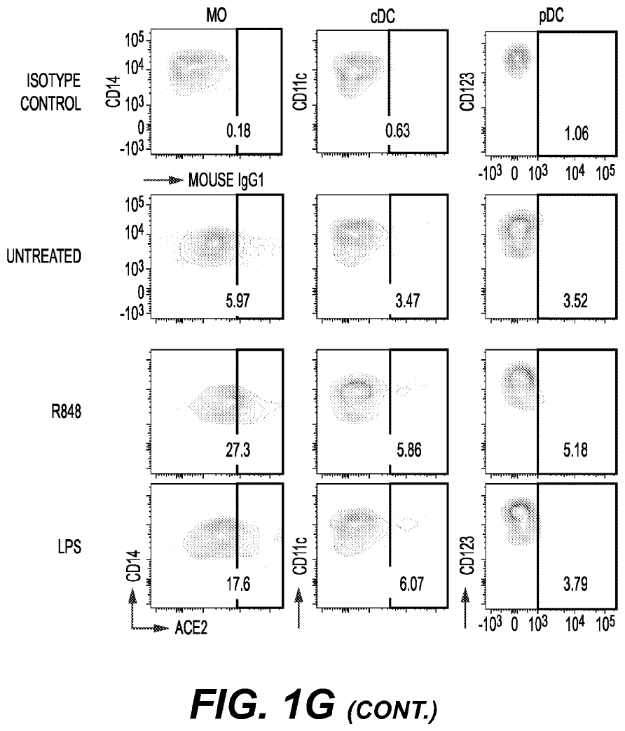

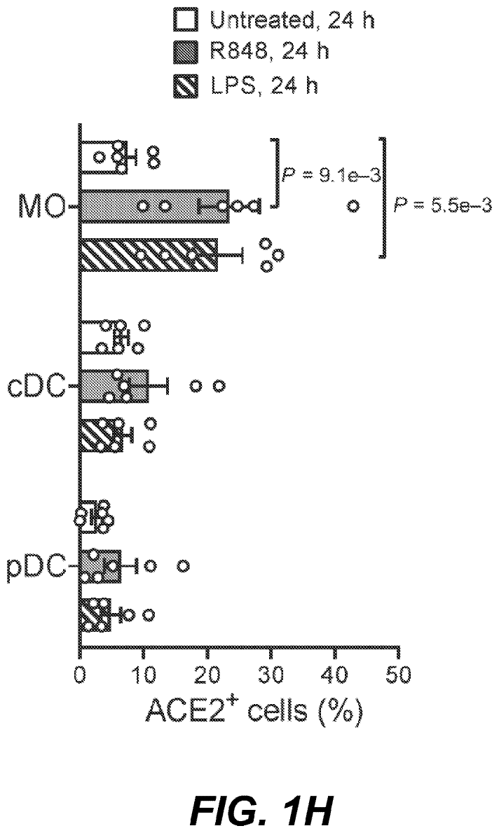

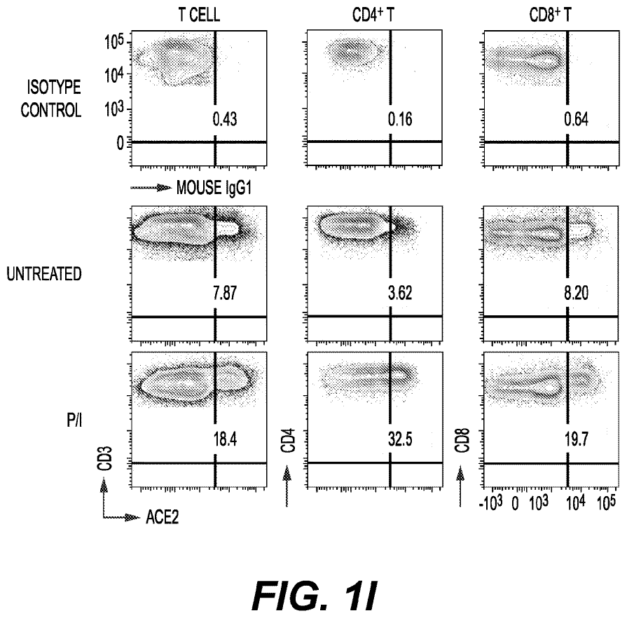

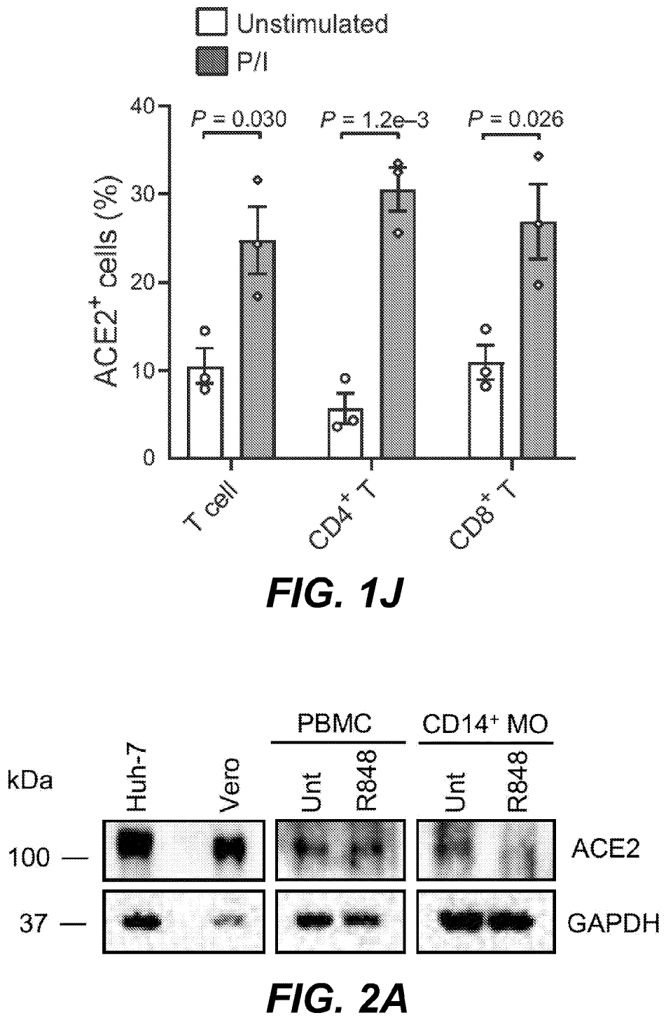

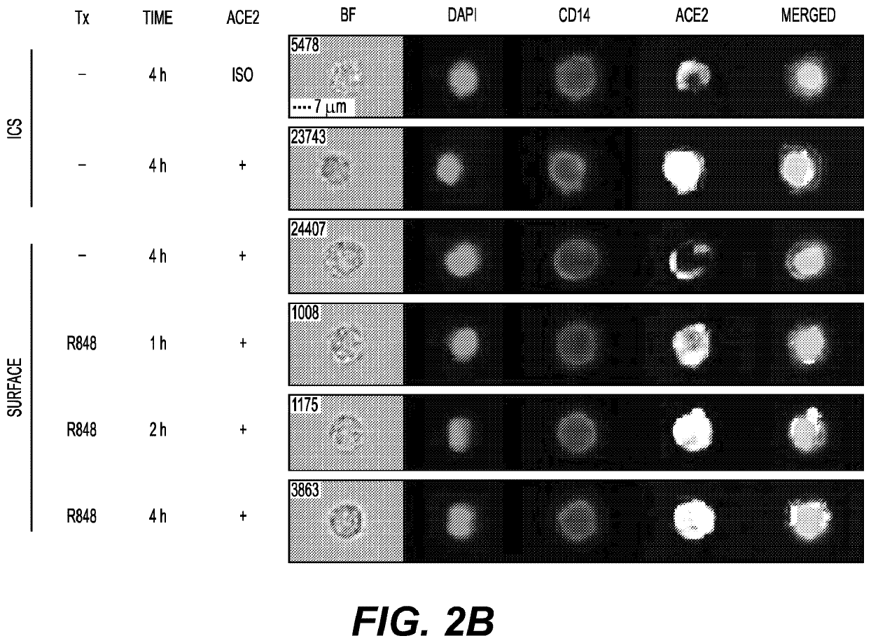

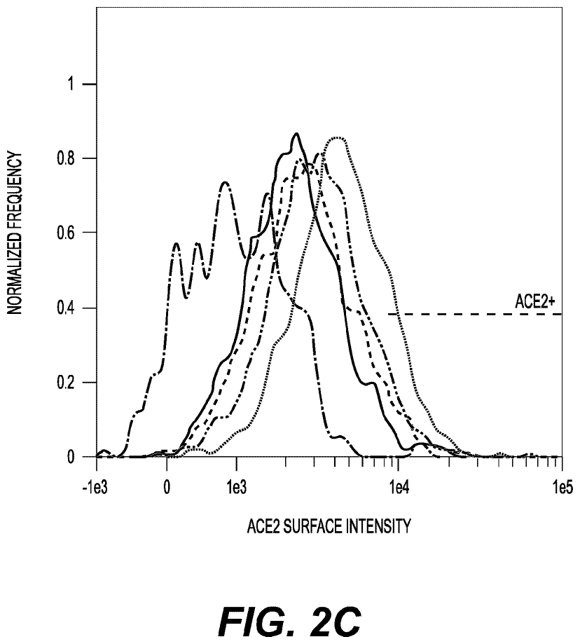

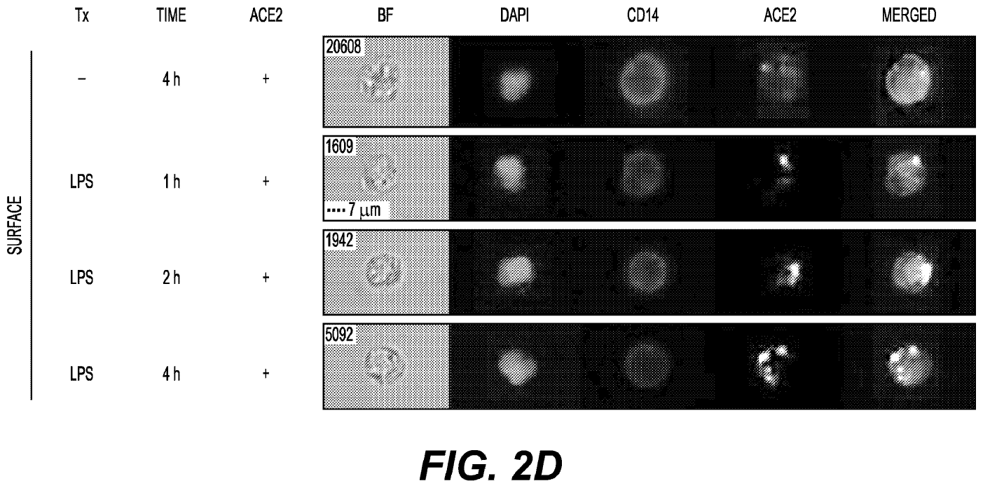

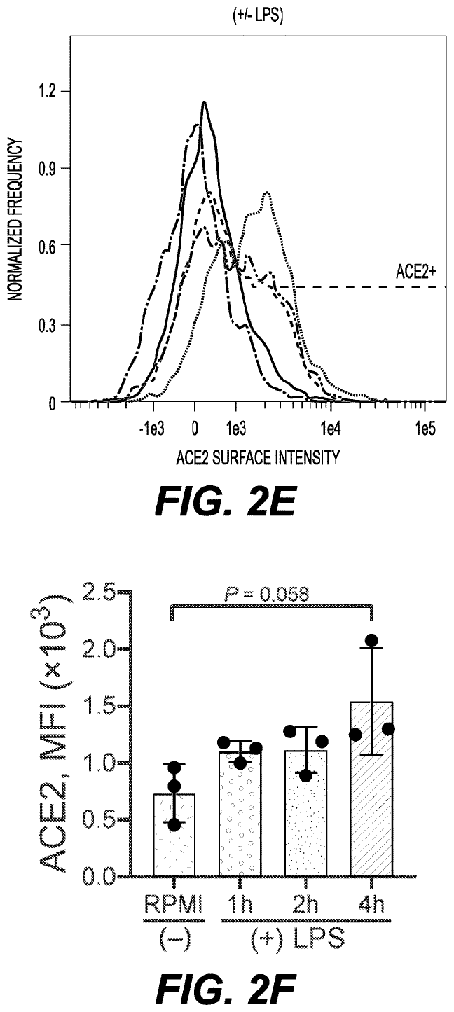

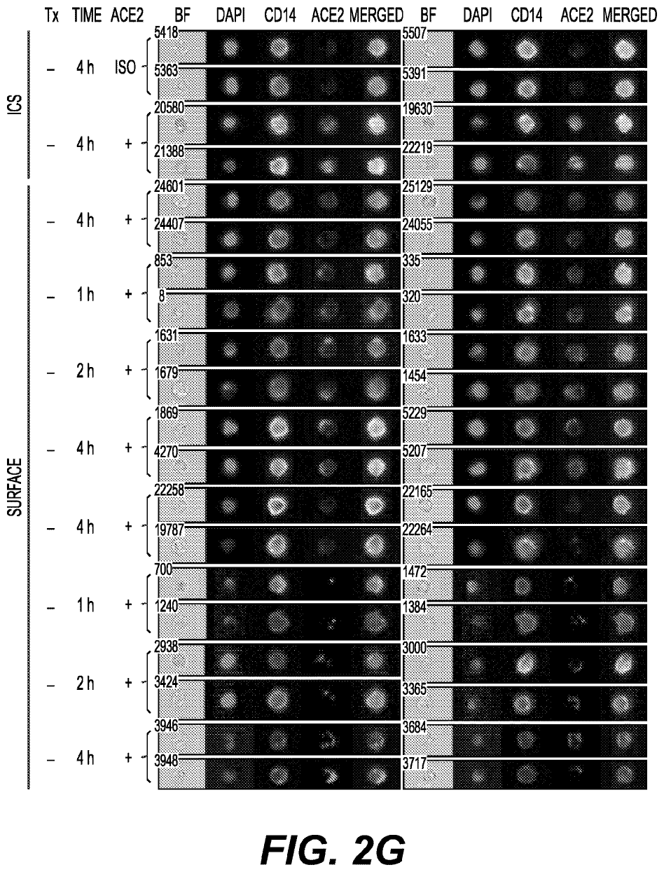

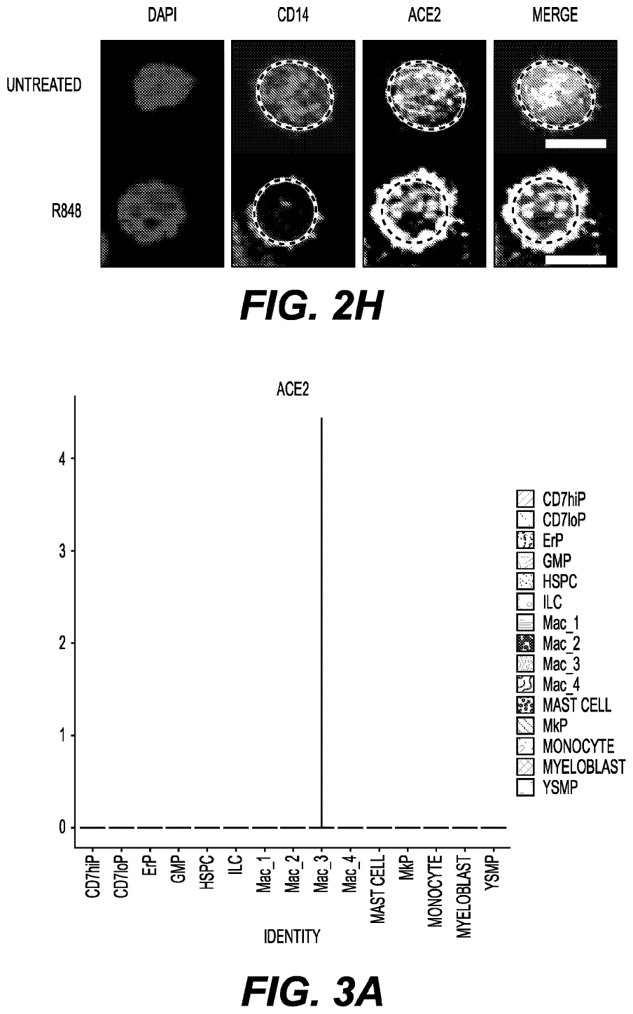

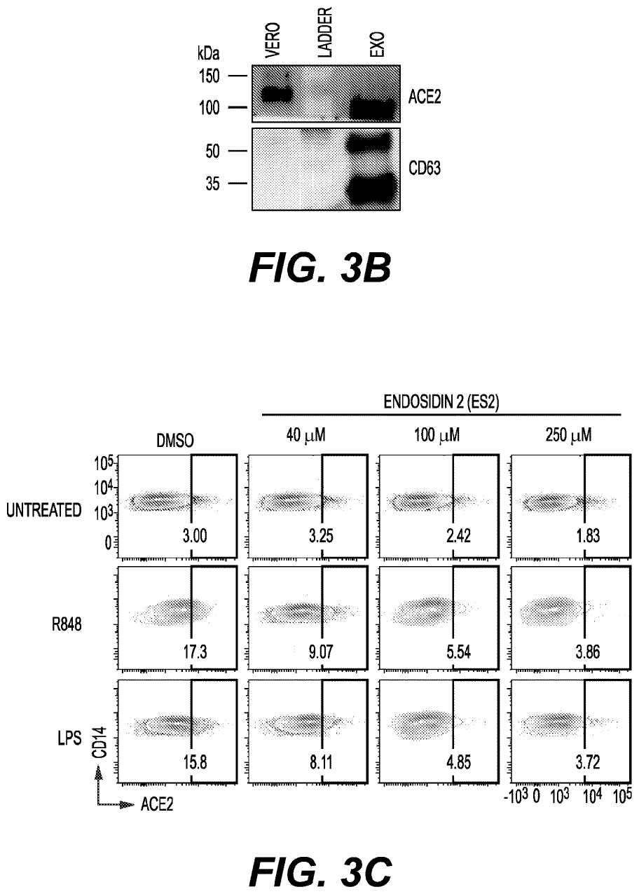

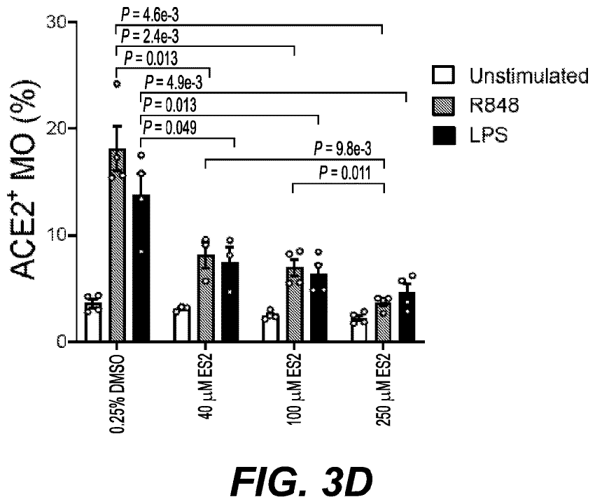

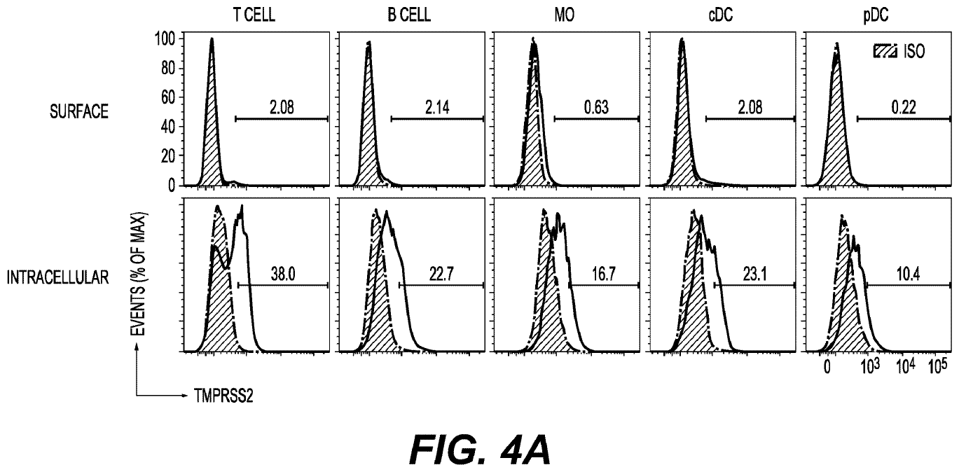

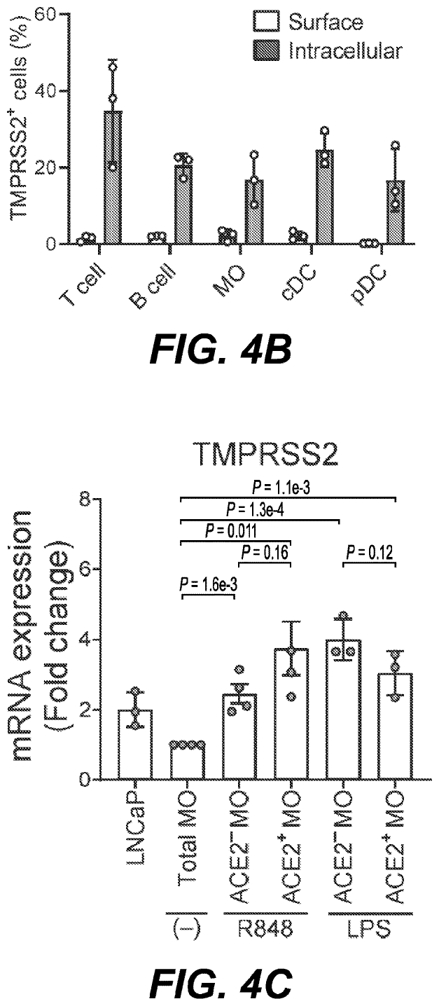

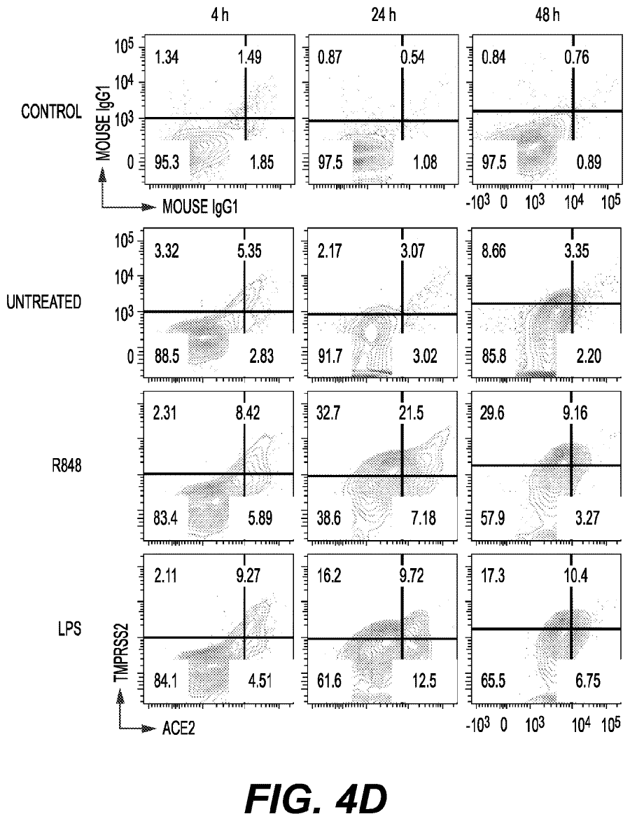

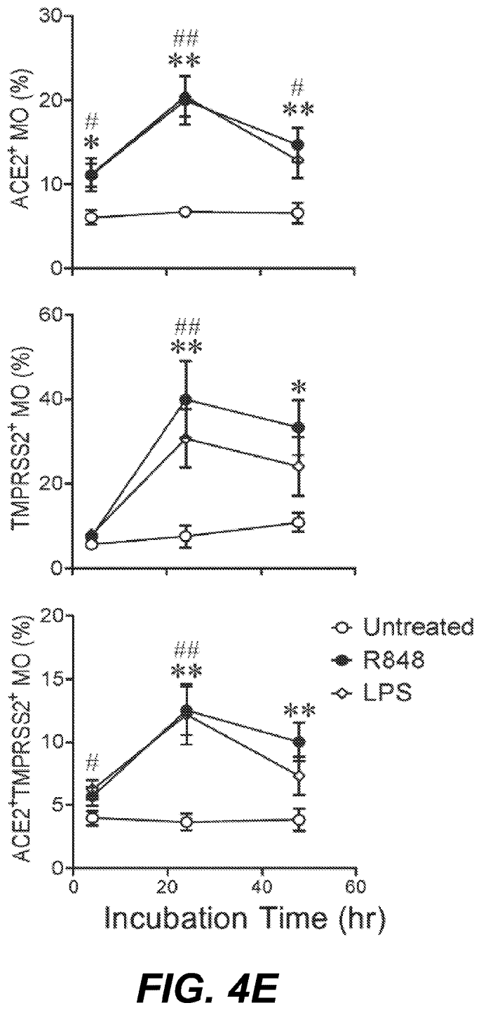

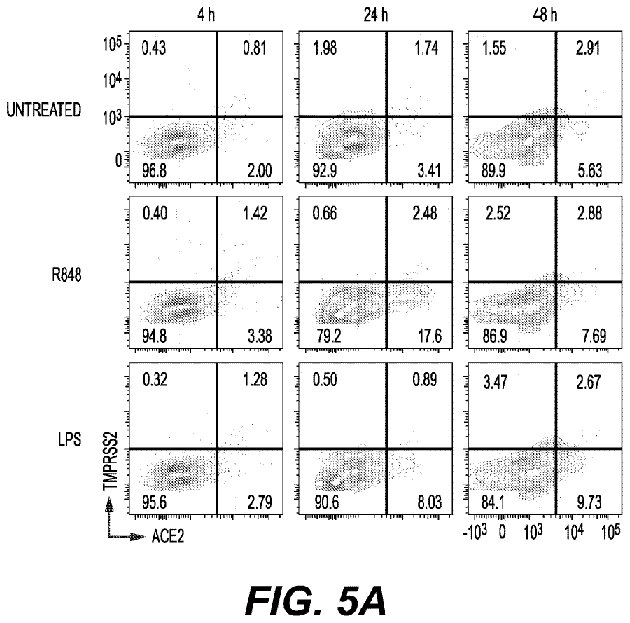

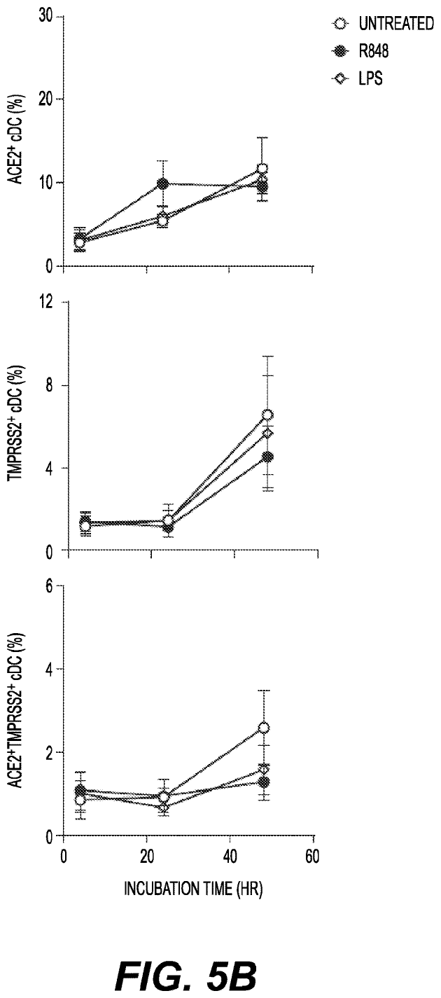

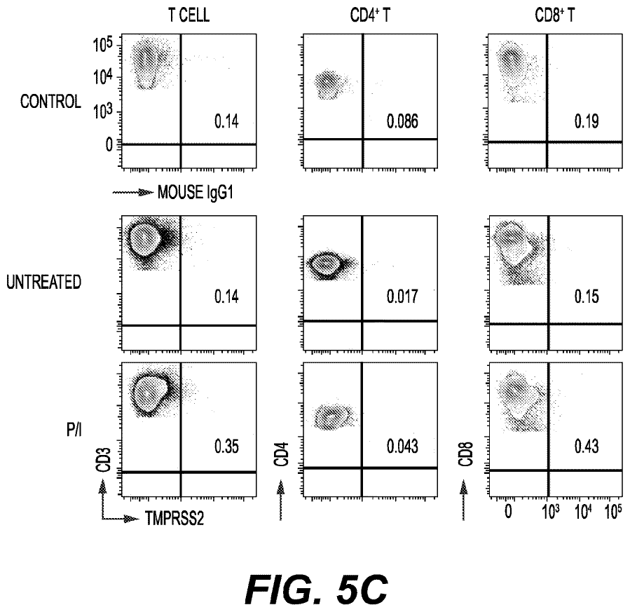

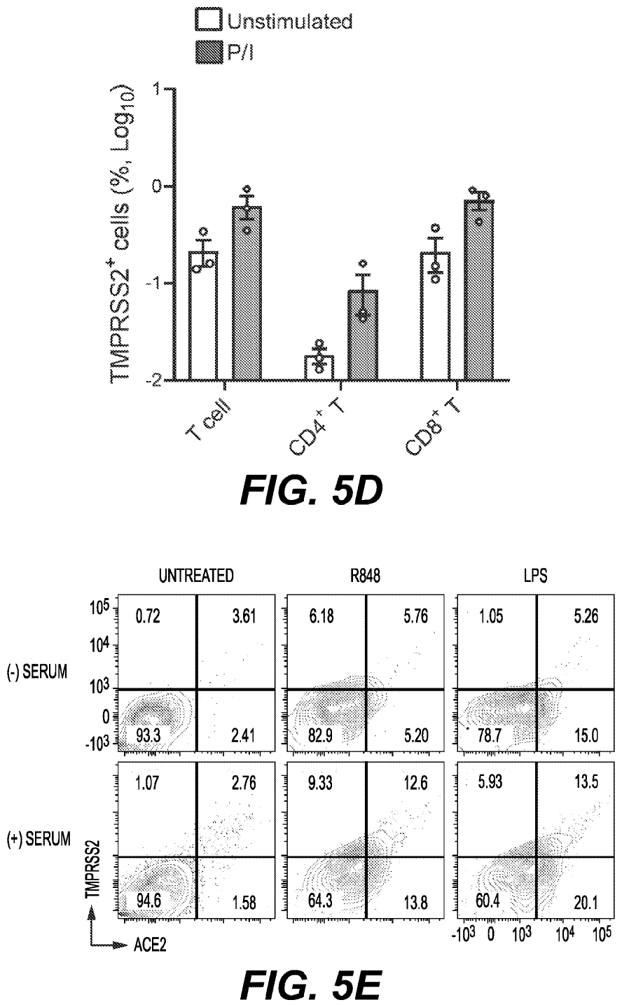

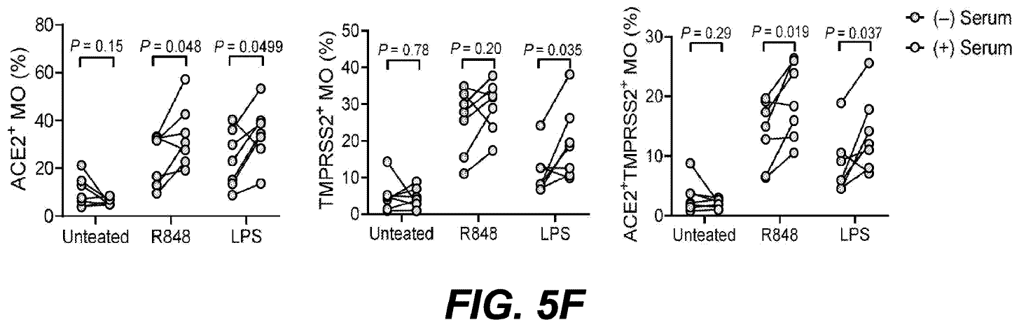

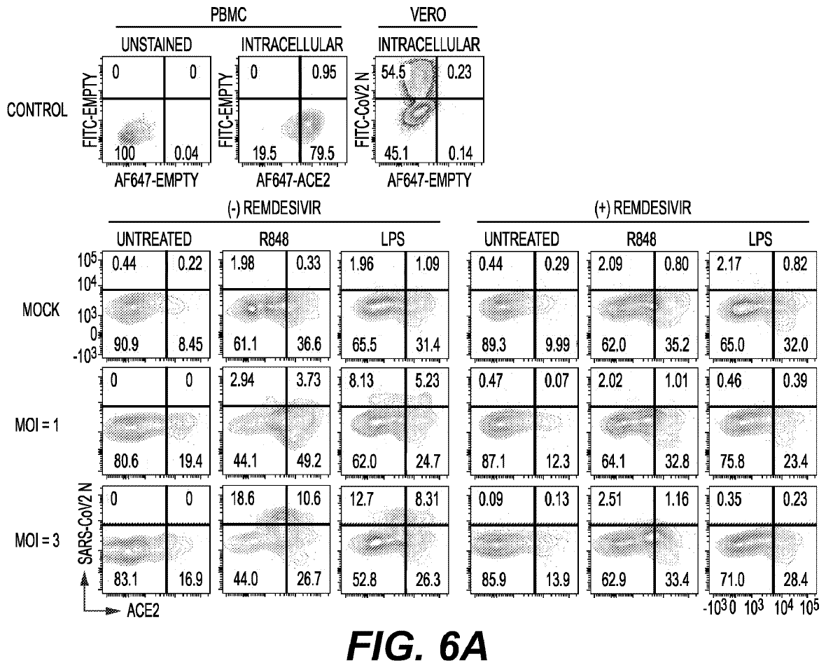

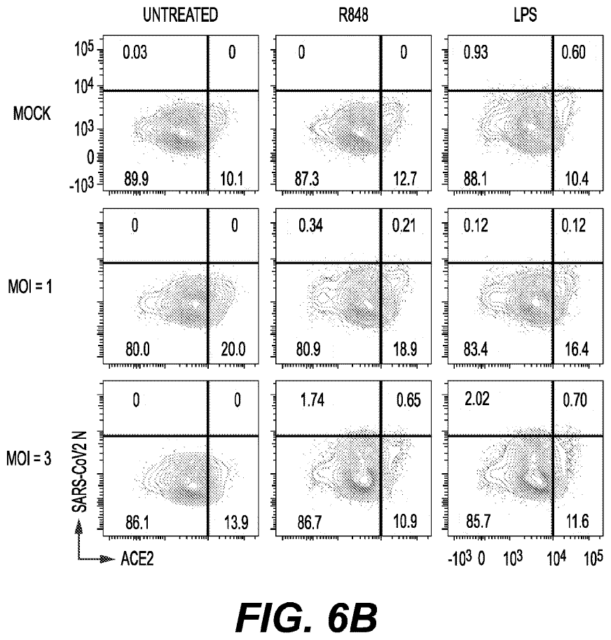

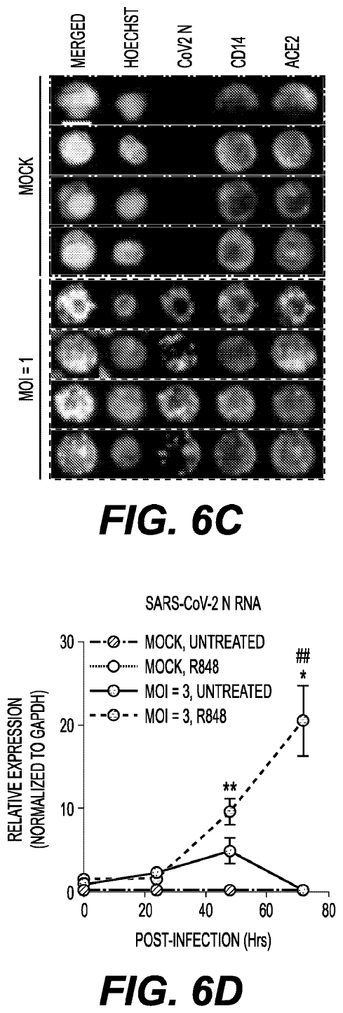

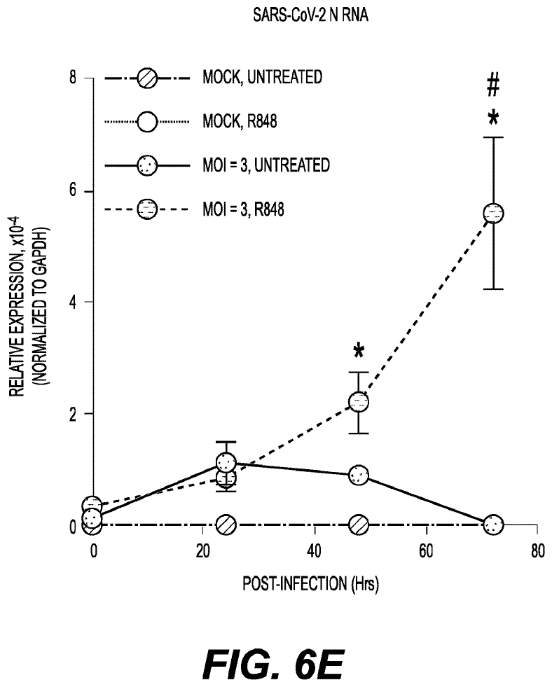

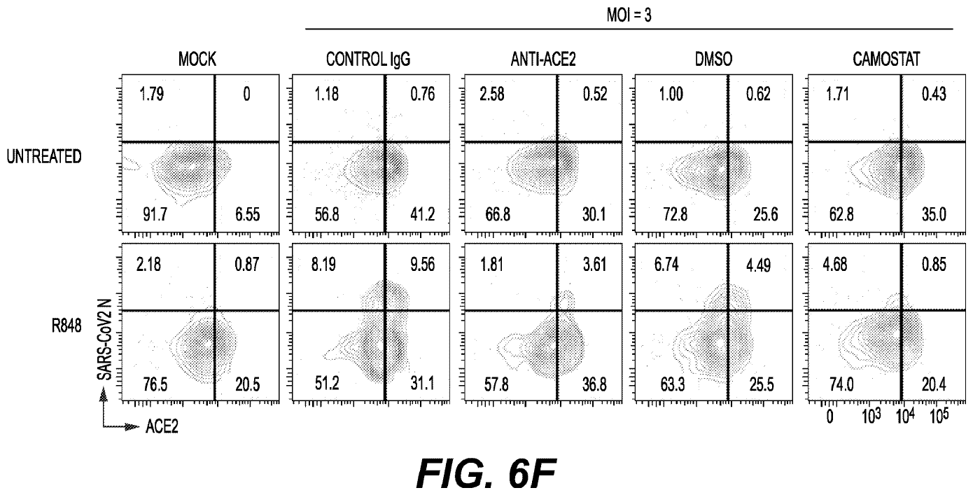

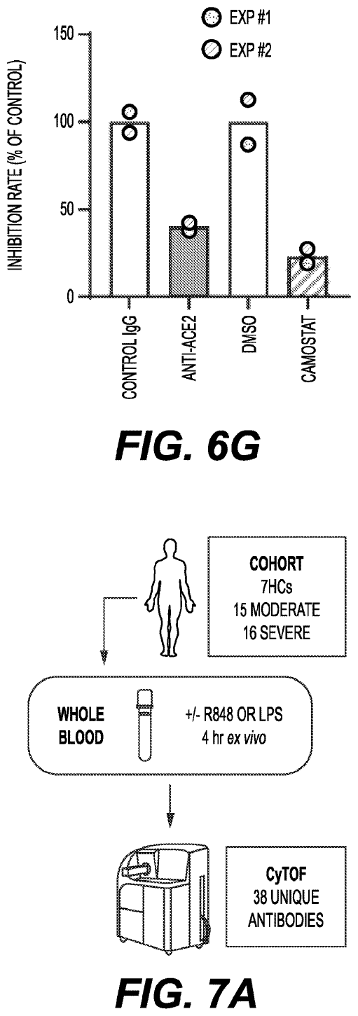

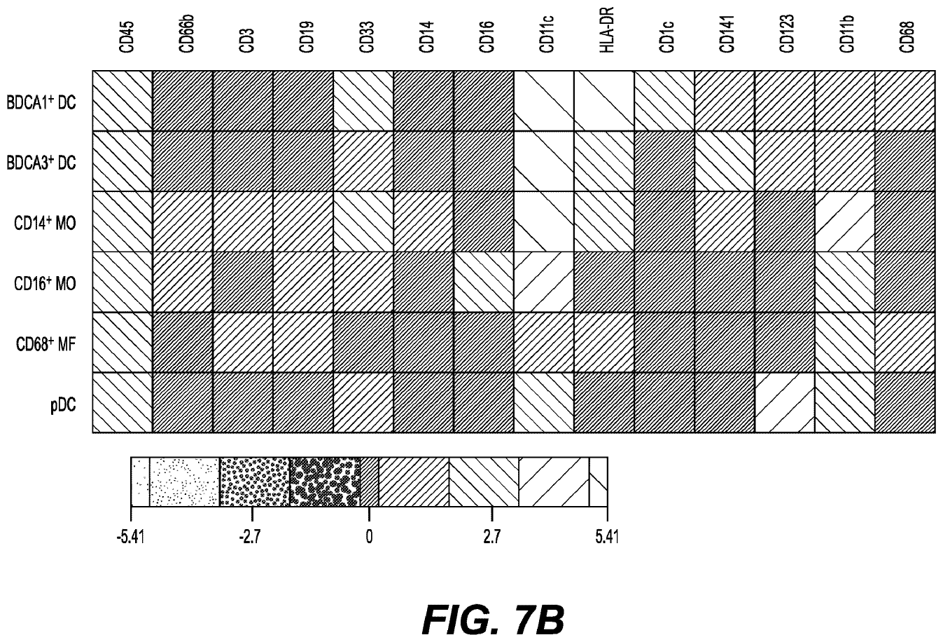

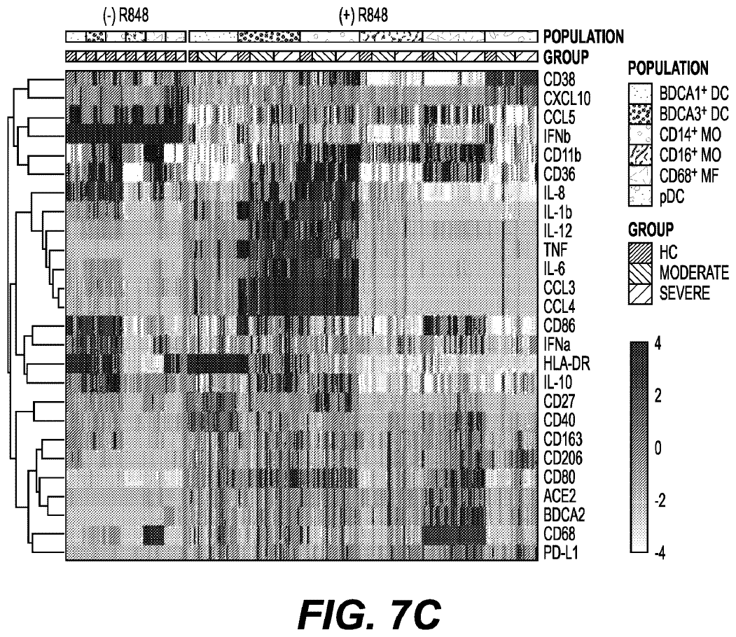

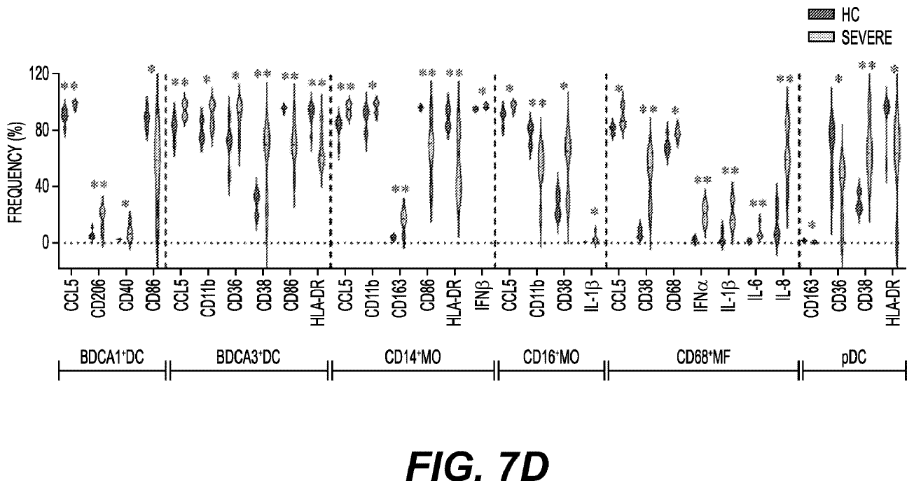

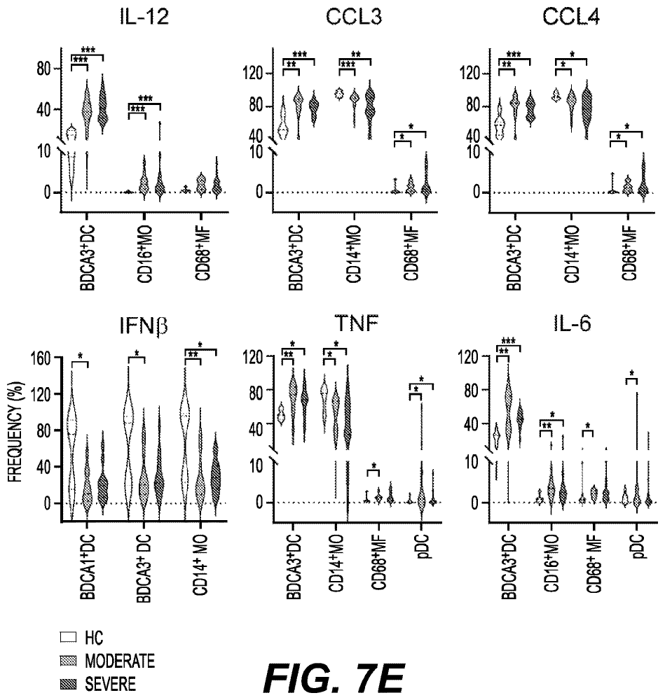

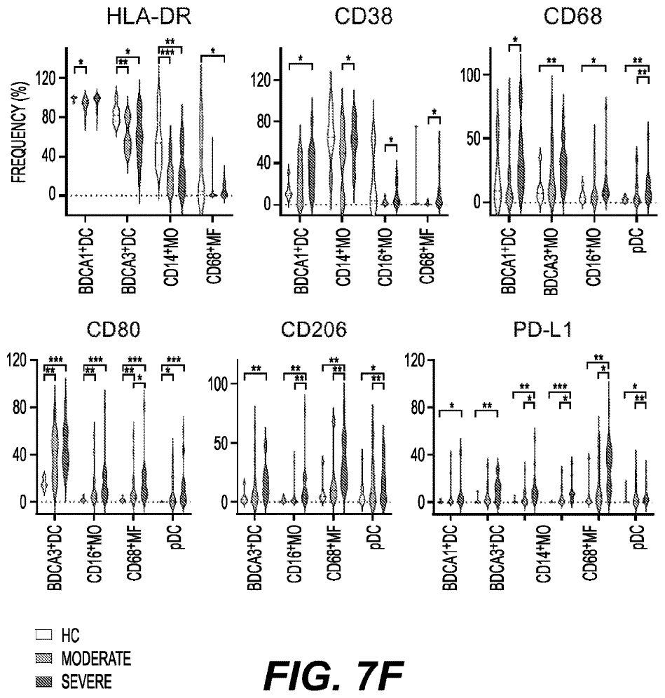

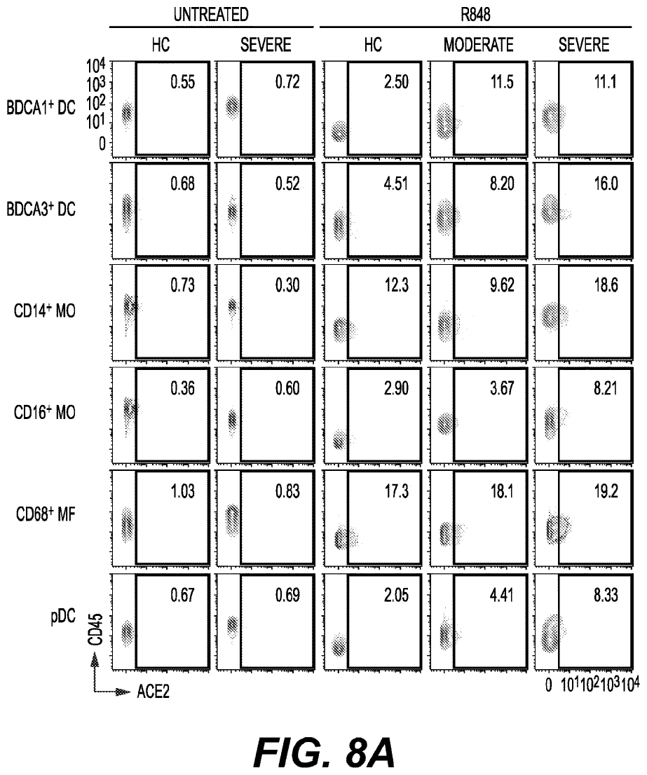

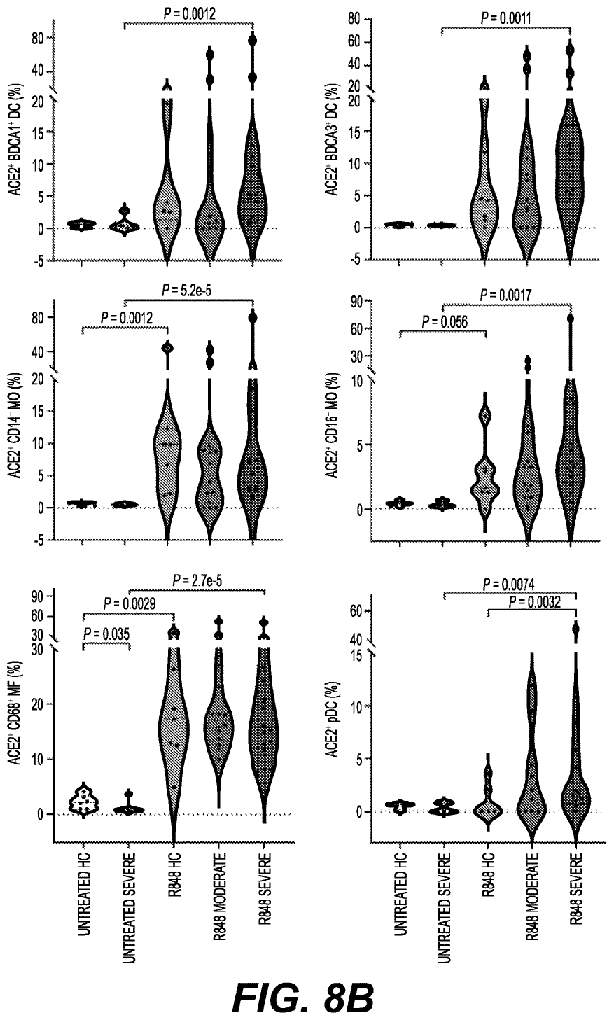

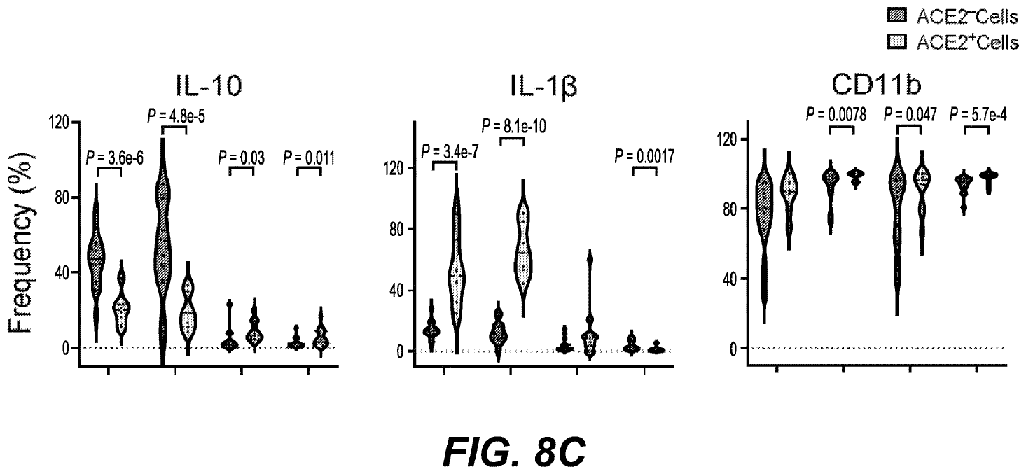

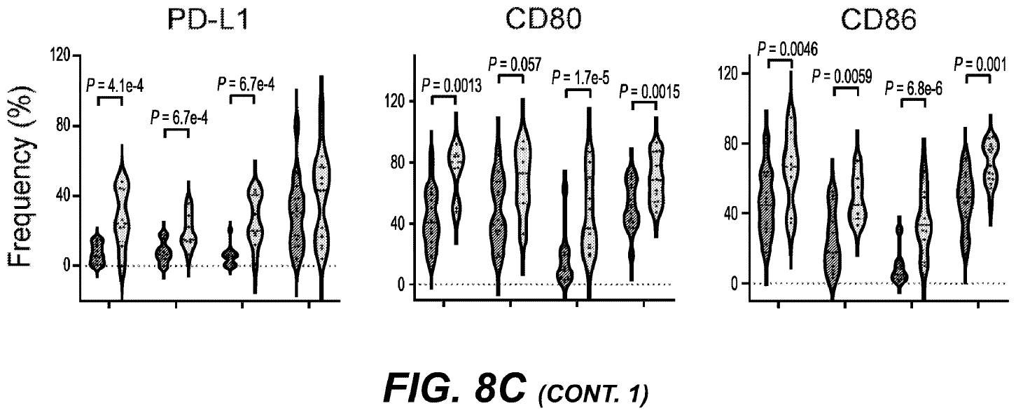

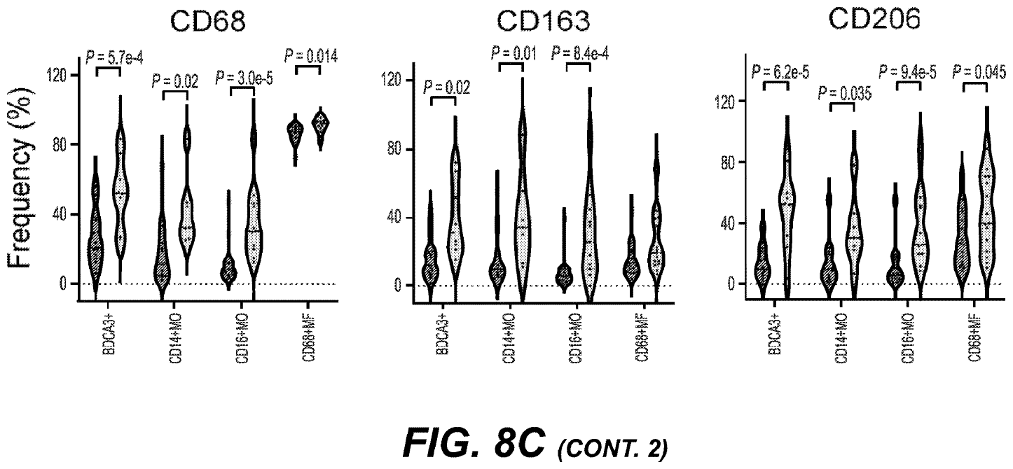

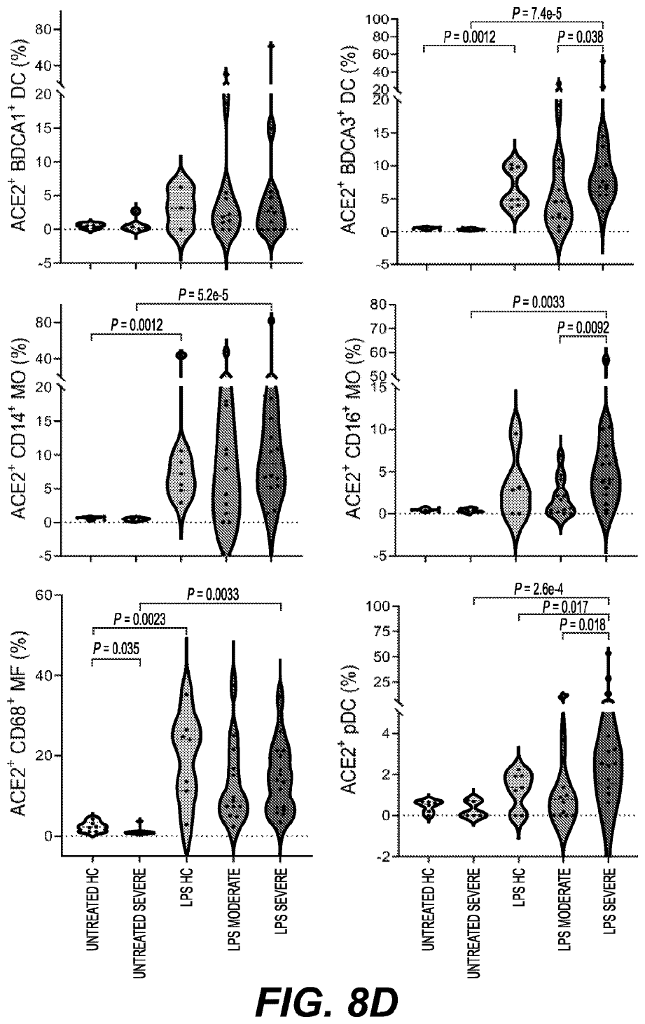

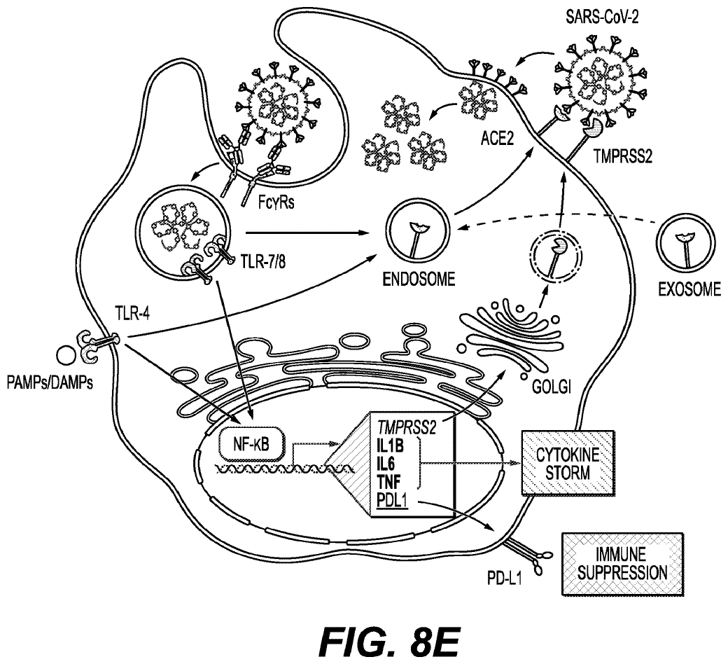

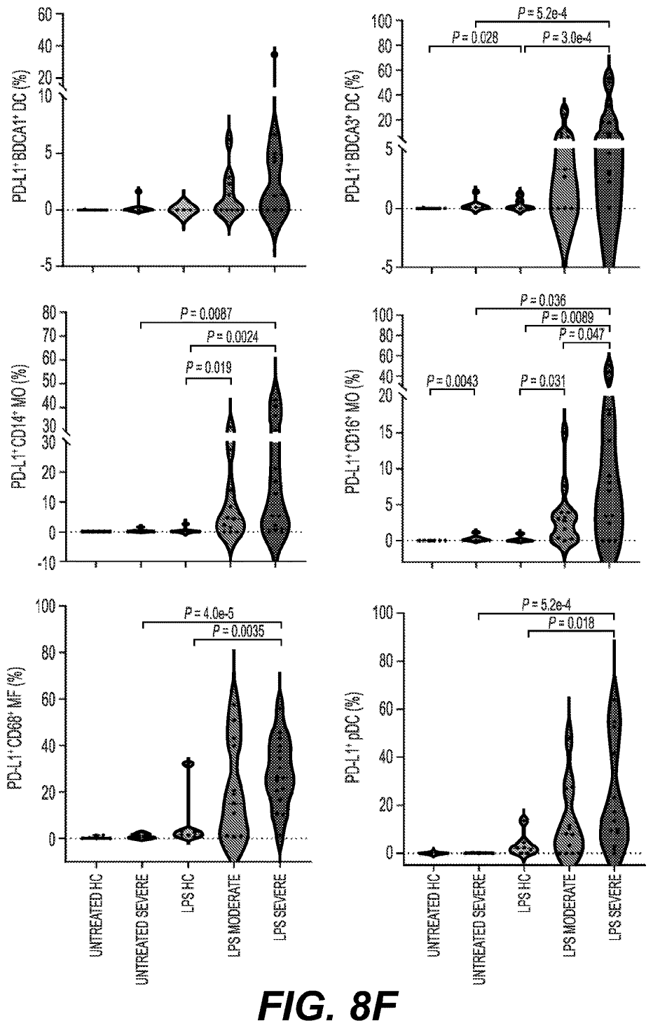

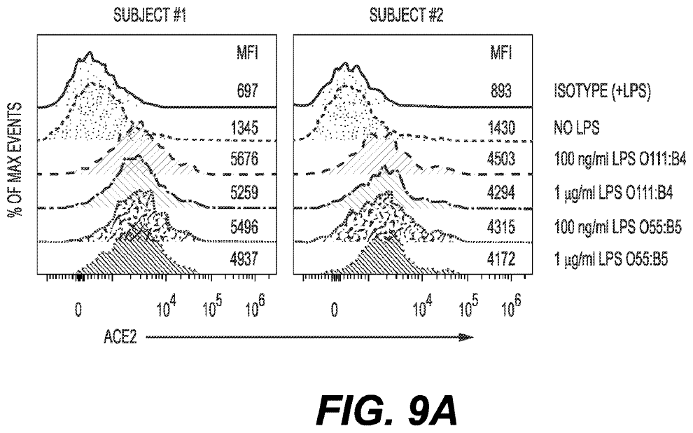

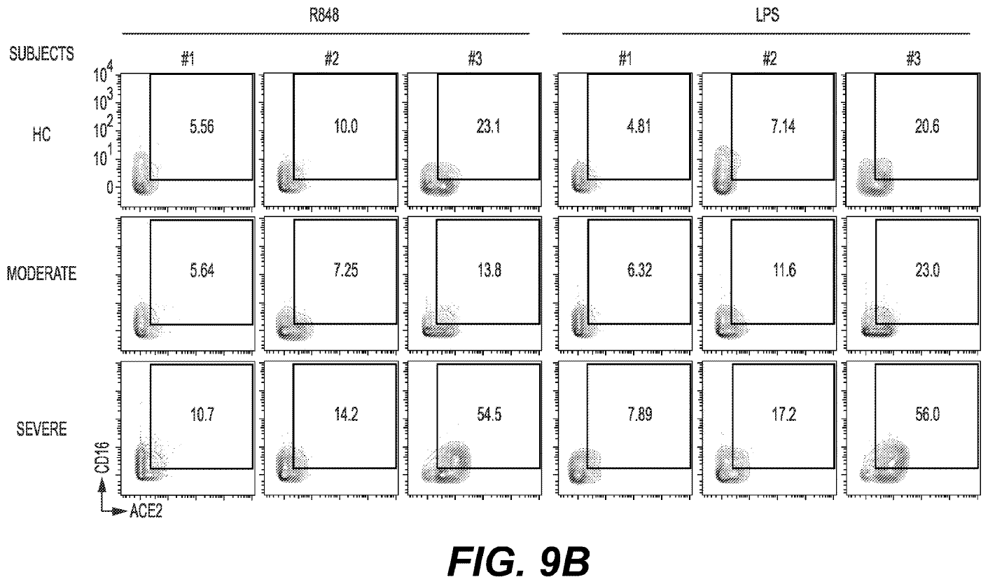

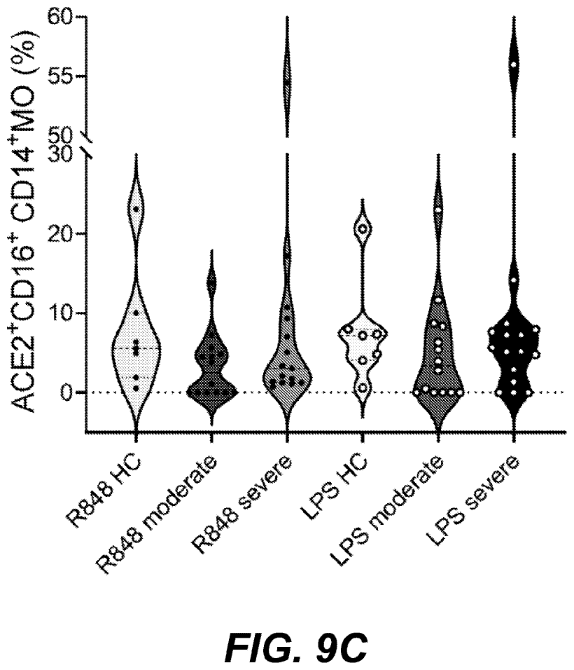

FIG. 1E shows ACE2 mRNA expression in monocytes upon ex vivo stimulation. PBMC samples were ex vivo cultured in the presence or absence of R848 or LPS for 4 h and stained with anti-CD14 and anti-ACE2 antibodies on cell surface. ACE2+co14+, ACE2-co14+ and total monocytes (MO) were sorted for qRT-PCR analysis. Relative expression of ACE2 mRNA in the indicted monocyte populations compared to that in human prostate adenocarcinoma LNCaP cell line, (N=3-4 biologically independent samples per group).

FIG. 1F shows ACE2 mRNA expression in T cells upon ex vivo stimulation. PBMCs were ex vivo cultured with PMA plus ionomycin for 4 h. ACE2+CD3+T, ACE2-co3+T and total CD3+ T cells were sorted for qRT-PCR analysis. Relative expression of ACE2 mRNA in the indicted T cell populations compared to that in LNCaP cells, (N=3 biologically independent samples per group).

FIG. 1G shows TMPRSS2 mRNA expression in T cells upon ex vivo stimulation. PBMCs were ex vivo cultured with PMA plus ionomycin for 4 h. ACE2+CD3+T, ACE2-co3+T and total CD3+ T cells were sorted for qRT-PCR analysis. Relative expression of TMPRSS2 mRNA in the indicated populations compared to that in the total T cells without treatment, (N=3 biologically independent samples per group).

FIG. 1H shows PBMCs cultured ex vivo with or without R484 or LPS for 24 h, followed by flow cytometry analysis of surface ACE2 in col 4+MO, cDCs, and pDCs. Mouse IgG1 antibody was used as the isotype control.

FIG. 11 shows the frequencies of indicated cell types expressing surface ACE2 as in (1H) (n=6 biologically independent samples/group). cDC, classical dendritic cells; pDC, plasmacytoid DCs; MO, monocytes; LPS, lipopolysaccharide.

FIG. 1J shows ACE2 surface expression in T cell populations from PBMCs. PBMCs were cultured ex vivo in the presence of PMA plus ionomycin (P/I) for 4 h, followed by flow cytometry analysis of surface ACE2 in total T cells and co4+ and cos+ T cells.

FIG. 1K shows ACE2 surface expression in T cell populations from PBMCs. The frequencies of the indicated T cell populations expressing surface ACE2 as in a (n=3 biologically independent samples/group).

FIG. 2A shows ACE2 protein levels in PBMCs and monocytes upon ex vivo stimulation with R848. PBMCs isolated from healthy donors were ex vivo cultured in the presence or absence of R848 for 24 h followed by CD14+ monocyte (MO) enrichment (>85% purity). A total of 60 μg protein from untreated (Unt) or R848-treated PBMCs and enriched monocytes were loaded to the gels for immunoblotting. Representative blot bands showing the expression of ACE2. GAPDH serves as a loading control.

FIG. 28 shows the rapid translocation of cytoplasmic ACE2 to the cell surface of co14+ monocytes after TLR activation. Imaging flow cytometry analysis of cytoplasmic and surface ACE2 protein in col 4+MO with or without R848 treatment up to 4 hrs. Each cell is represented by the row of images that include bright field (BF), DAPI (purple), CD14 (turquoise), ACE2 (red), and the overlapping image merged with DAPI, CD14, and ACE2.

FIG. 2C shows a histogram ofACE2 intensity on the cell surface of CD14+ monocytes treated with R848. Grey line represents the isotype control group (Iso).

FIG. 20 shows a column graph that indicates MFI ofACE2 surface intensity at other indicated conditions. (n=3 biologically independent samples/group).

FIG. 2E shows the translocation of cytoplasmic ACE2 protein to the cell surface of CD14+ monocytes upon exvivo stimulation with LPS. Imaging flow cytometry analysis of cytoplasmic and surface ACE2 protein in CD14+ monocytes treated (Tx) with orwithout LPS up to 4 hrs. Each cell is represented by the row of images that include bright field (BF), DAPI (purple), CD14 (turquoise), ACE2 (red), and the overlapping image merged with DAPI, CD14, and ACE2.

FIG. 2F shows a histogram of ACE2 intensity on the cell surface of CD14+ monocytes treated with LPS. Grey line represents the isotype control group (Iso).

FIG. 2G shows a column chart of the mean fluorescence intensity (MFI) of surface ACE2 at other indicated conditions (n=3 biologically independent samples/group).

FIG. 2H shows imaging flow cytometry of intracellular and surface ACE2 in peripheral blood monocytes. PBMCs were treated (Tx) with or without R848 or LPS up to 4 h and analyzed for ACE2 expression on the cell surface or intracellularly (ICS) in col 4+ monocytes. Each cell is represented by the row of images that include bright field (BF), DAPI (purple), CD14 (turquoise), ACE2 (red), and the overlapping image merged with DAPI, CD14, and ACE2. Four cells are shown for each condition.

FIG. 21 shows representative confocal microscopy of ACE2 protein in co14+ monocytes with or without R848 treatment for 24 h. Bars, 10 μm. Dashed lines show location of cell membrane. Images show cells from two healthy donors with similar results.

FIG. 3A shows an ACE2 transcriptomic analysis of CD45+ haematopoietic cells in tissues from human embryos. CD45+CD235a− haematopoietic cells were collected from yolk sac, head, liver, blood, skin and lung between Carnegie stage 11-23 (C11-C23) for STRT-seq. Violin plot showing mRNA expression of ACE2 by 15 identified clusters from the public dataset GSE133345. CD7hi/loP, CD7hi/lo progenitors; ErP, erythroid progenitors; GMP, granulocyte-monocyte progenitors; HSPC, haematopoietic stem and progenitor cells; ILC, innate lymphoid cells; Mac, macrophage; MkP, megakaryocyte progenitors; YSMP, yolk sac-derived myeloid-biased progenitors.

FIG. 38 shows an immunoblot of ACE2 and CD63 expression in exosomes isolated from plasma of healthy donors. The monkey kidney epithelial cell line Vero was used as a positive control.

FIG. 3C shows a flow cytometry analysis of ACE2 surface expression in PBMCs pre-treated with the indicated concentrations of endosidin 2 (ES2) or DMSO for 1-2 h before ex vivo incubation with R484, LPS, or medium alone for 4 hrs.

FIG. 30 shows the frequency of ACE2+MO for data shown in (3C). Bars represent mean±SEM of biologically independent samples, (n=3/group for 40 μM ES2 condition; n=4/group for all other conditions).

FIG. 4A shows concurrent expression of ACE2 and TMPRSS2 on the cell surface of monocytes after TLR4/7/8 stimulation. Representative flow cytometric analysis of TMPRSS2 protein expression in T cells, B cells, co14+MO, cDCs, and pOCs from PBMCs isolated from healthy donors. Mouse IgG1 antibody (gray tinted) was used as isotype (Iso) control.

FIG. 48 shows frequency of cells with cell surface and cytoplasmic TMPRSS2 expression as in (4A), (n=3 biologically independent samples/group).

FIG. 4C shows relative TMPRSS2 mRNA expression in indicated cell types compared to expression in total MOs without treatment. A human prostate adenocarcinoma cell line LNCaP was used as the positive control.

FIG. 40 shows flow cytometry analysis of ACE2 and TMPRSS2 cell surface expression in PBMCs cultured ex vivo with R484, LPS, or medium alone for 4, 24, and 48 hrs. Mouse IgG1 antibodies were used as the isotype controls.

FIG. 4E shows frequency of ACE2+MO, TMPRSS2+MO, and ACE2+ TMPRSS2+MO for each condition in d. Bars represent mean±SEM of biologically independent samples, (n=6/group at 4 h and 24 h; n=5/group at 48 h; and n=4/group at 72 h). * indicates comparisons between R848-treated and untreated groups; #indicates comparison of LPS-treated and untreated groups. * or #, P<0.05; ** or ##, P<0.01.

FIG. SA shows surface expression of ACE2 and TMPRSS2 in cDC upon exposure to R848 and LPS. PBMCs were ex vivo cultured with R848, LPS or medium alone for 4 hrs, 24 hrs, and 48 hrs. Representative flow cytometric analysis of surface expression of ACE2 and TMPRSS2 in cDC.

FIG. SB shows the frequencies of ACE2+ cDC, TMPRSS2+ cDC and ACE2+ TMPRSS2+ cDC for each condition in a. Bars represent mean±s.e.m. of biologically independent samples (n=6/group at each time point).

FIG. SC shows the surface expression of TMPRSS2 in circulating T cells upon ex vivo stimulation. PBMCs were ex vivo cultured with PMA plus ionomycin for 4 h. Representative flow cytometric analysis of surface expression of TMPRSS2 in CD4+, CD8+ and total T cells.

FIG. SD shows the frequencies of the indicated populations expressing surface TMPRSS2 as in FIG. SC. Bars represent mean±s.e.m. of biologically independent samples (n=3/group).

FIG. SE shows the surface expression of ACE2 and TMPRSS2 in monocytes upon exposure to R848 and LPS in the presence or absence of serum. PBMCs were ex vivo cultured with RPM11640 medium in the presence or absence of 10% FBS and with R848, LPS, or medium alone for 24 hrs. Representative flow cytometric analysis of surface expression of ACE2 and TMPRSS2 in monocytes (MO).

FIG. SF shows the frequencies of ACE2+MO, TMPRSS2+MO and ACE2+ TMPRSS2+MO for each condition in SE. Each dot represents one biologically independent sample, (n=6/group).

FIG. GA shows the infection of ACE2+co14+ monocytes by SARS-CoV-2 upon TLR4/7/8 activation. PBMCs were cultured with R848, LPS, or medium alone with or without 100 nM remdesivir for 2 h before infection. Pretreated cells were infected with SARS-CoV-2 (MOI=1 or 3) or mock-infected for 24 h in the presence of stimuli and/or inhibitor. Flow cytometry analysis of col 4+ monocytes from mock-infected and SARS-CoV-2-infected cells showing cell surface ACE2 (Alexa Fluor 647-labeled antibody) and intracellular SARS-CoV-2 nucleocapsid (N) protein (FITC-labeled antibody). FACS channels with no staining are shown as “empty”. Data were collected pooled PBMCs from 3 healthy donors (controls). Two independent infection experiments were performed with similar results.

FIG. 68 shows that cDC are impermissive to SARS-CoV-2 upon exposure to R848 and LPS. PBMCs were cultured in the presence of R848 or LPS or medium alone for 2 h prior to infection with SARS-CoV-2 at MOI=1 or 3 for 24 h. Flow cytometry analysis of the surface ACE2 and intracellular SARS-CoV-2 nucleocapsid (N) protein in cDC from mock-infected and virus-infected cells. Data were collected from a pool of PBMCs from 3 healthy control samples. Two independent infection experiments were performed with similar results.

FIG. GC shows confocal fluorescence microscopy of R848-stimulated PBMCs infected with SARS-CoV-2 as in FIG. 6A and stained with Hoechst 33342 (nuclear stain) and fluorescent antibodies against SARS-CoV-2 N protein, CD14, and ACE2. Representative images of cells from mock and infected groups. Scale bar, 5 μm.

FIG. GD shows a graph of SARS-CoV-2 N RNA expression as measured by qRT-PCR in PBMCs pretreated with R848 or medium alone for 2 h and infected with SARS-CoV-2 at MOI=3 or mock-infected for an additional 2 h. Cells were washed then cultured with fresh medium (10% serum) for indicated time points before mRNA extraction. * indicates comparisons between 0 h and other time points for R848-treated groups with viral infections. #indicates comparison of R848-treated and untreated groups with viral infections at the same time points. * or #, P<0.05; ** or ##, P<0.01.

FIG. GE shows a graph of SARS-CoV-2 sgRNA expression as measured by qRT-PCR in the cells as treated in FIG. 60.

FIG. GF shows the flow cytometry of cell surface ACE2 and intracellular SARS-CoV-2 N protein in CD14+ monocytes from PBMCs cultured with R848 or medium alone with or without 2 μg/ml anti-ACE2 antibody or goat IgG control, 50 μM camostat mesylate, or 2% DMSO (vehicle) for 2 h and then infected with SARS-CoV-2 at MOI=3 or mock-infected for 24 h in the presence of stimuli and/or antibody/inhibitor.

FIG. GG shows the percentage of ACE2+ CoV-2 W monocytes in antibody or inhibitor-treated groups compared to control groups. Each dot represents pooled PBMCs from 3 healthy donors (controls). Two independent infection experiments were performed with similar results.

FIG. 7A shows the pipeline for processing, treatment, and analysis of blood samples from healthy controls (HCs) and patients with moderate and severe COVID-19. Whole blood cells were stimulated with or without R848 for 4 h ex vivo before staining with antibody mixture for mass cytometry (CyTOF).

FIG. 78 shows a heat map of median intensity of the indicated lineage markers for each myeloid population after gating of myeloid populations in whole blood upon ex vivo stimulation. The color scale was obtained after calculating transformed ratio of medians by Table's Minimum using values of X-Axis channels in Cytobank platform. MO, monocytes; MF, macrophages.

FIG. 7C shows the hyperinflammatory states of blood myeloid cells from COVID-19 patients before and after ex vivo stimulation with R848.

FIG. 70 shows violin plots of the frequencies of myeloid populations expressing indicated markers from HCs (n=7) and cells from patients with severe COVI0-19 (n=7) without R848 treatment.

FIG. 7E shows violin plots of frequencies of myeloid populations stimulated ex vivo with R848 expressing indicated cytokines and chemokines from HCs (n=7) and COVI0-19 patients with moderate (n=15) or severe (n=16) diseases. *, P<0.05; **, P<0.01; ***, P<0.001.

FIG. 7F shows violin plots of frequencies of myeloid populations stimulated ex vivo with R848 expressing surface markers from HCs (n=7) and COVID-19 patients with moderate (n=15) or severe (n=16) diseases. *, P<0.05; **, P<0.01; ***, P<0.001.

FIG. SA shows ACE2 surface translocation upon ex vivo R848 stimulation is positively associated with proinflammatory responses and P0-L1 expression in blood myeloid cells from COVI0-19 patients. Mass cytometry analysis of cell surface ACE2 expression in blood myeloid populations from untreated samples (n=7 HC; n=7 severe COVI0-19) and R848-treated samples (n=7 HC; n=15 moderate COVI0-19; and n=16 severe COVID-19).

FIG. SB shows violin plots of frequencies of the myeloid compartment expressing surface ACE2 as shown in FIG. SA.

FIG. SC shows the frequencies of ACE2+ and ACE2− cells (based on ACE2 surface expression) within the myeloid compartment expressing the indicated markers in cells from severe COVI0-19 patients.

FIG. SO shows the surface expression of ACE2 in circulating myeloid populations upon LPS ex vivo stimulation. Whole blood samples were ex vivo stimulated in the presence or absence of LPS for 4 h, and then stained with metal-conjugated antibodies for mass cytometry analysis. Violin plots showing the frequencies of myeloid compartment expressing surface ACE2 from untreated samples (n=7 healthy controls (HC); n=7 severe COVID-19) as well as R848-treated samples (n=7 HC; n=15 moderate COVI0-19; and n=16 severe COVID-19) of cohort.

FIG. SE shows a graphic summary of SARS-CoV-2 infection in monocytes co-expressing surface ACE2 and TMPRSS2 upon TLR4/7/8 activation. ACE2 is taken up by monocytes from ACE2-containing exosomes and stored in the early endosome at steady state. TLR7/8 activation triggered by endocytosis of SARS-CoV-2 or TLR4 activation triggered by viral proteins or host-derived danger signals released during infection stimulates downstream TLR signaling pathways to enhance gene expression of TMPRSS2, proinflammatory cytokines, and PD-L1, which drive the cytokine storm and promote immune suppression. TLR4/7/8 activation also induces ACE2 translocation through endosomal trafficking to the cell membrane. Translocated ACE2 and newly synthesized TMPRSS2 at the cell surface facilitates SARS-CoV-2 viral entry and active replication in monocytes. Image generated with 8ioRender.

FIG. SF shows the surface expression of PD-L1 in circulating myeloid populations upon LPS ex vivo stimulation. Whole blood samples were ex vivo stimulated in the presence or absence of LPS for 4 h, and then stained with metal-conjugated antibodies for mass cytometry analysis. Violin plots showing the frequencies of myeloid compartment expressing surface PD-L1 from untreated samples (n=7 healthy controls (HC); n=7 severe COVID-19) as well as R848-treated samples (n=7 HC; n=15 moderate COVID-19; and n=16 severe COVID-19) of cohort.

FIG. 9A shows the inducible ACE2 surface expression in co14+ monocytes upon LPS ex vivo stimulation. P8MCs from two healthy subjects were ex vivo stimulated in the presence of LPS 0111:84 or LPS 055:85 at the indicated concentrations or medium alone for 16 h followed by flow cytometry analysis. Histogram showing ACE2 expression on the cell surface of co14+ monocytes from the treated samples. Mouse IgG1 served as an isotype control. Mean fluorescence Intensity (MFI) of ACE2 shown on the right side of each panel. Experiments were performed independently twice with similar results.

FIG. 98 shows the co-expression of ACE2 and CD16 in co14+ monocytes upon R848 or LPS ex vivo stimulation. Whole blood samples were exvivo stimulated inthe presence or absence of R848 or LPS for 4 h, and then stained with metal-conjugated antibodies for mass cytometry analysis. Mass cytometry analysis of ACE2 and CD16 expression on the cell surface of co14+ monocytes from R848 or LPS-treated (n=7 HC; n=14 moderate COVID-19; and n=15 severe COVID-19) samples.

FIG. 9C shows co-expression of ACE2 and CD16 in co14+ monocytes upon R848 or LPS ex vivo stimulation. Whole blood samples were ex vivo stimulated in the presence or absence of R848 or LPS for 4 h, and then stained with metal-conjugated antibodies for mass cytometry analysis. Violin plots of the frequencies ofACE2+co15+ cells within col 4+ monocytes as shown in FIG. 98.

### DETAILED DESCRIPTION

### Definitions

Unless defined otherwise, all technical and scientific terms used herein have the same meaning as commonly understood by one of ordinary skill in the art to which this disclosure belongs. Accordingly, the following terms are intended to have the following meanings:

As used in the specification and claims, the singular form “a”, “an” and “the” includes plural references unless the context clearly dictates otherwise.

A “subject,” as used herein, can refer to any animal which is subject to a viral infection, e.g., a mammal, such as an experimental animal, a farm animal, pet, or the like. In some embodiments, the animal is a primate, preferably a human. As used herein, the terms “subject” and “patient” are used interchangeably. The terms “subject” and “patienf refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment the subject is a “human”.

As used herein, “treatment” and “treating”, are used interchangeably herein, and refer to an approach for obtaining beneficial or desired results including, but not limited to, therapeutic benefit. By therapeutic benefit is meant eradication or amelioration of the underlying disease being treated. Also, a therapeutic benefit is achieved with the eradication or amelioration of one or more of the physiological symptoms associated with the underlying disease such that an improvement is observed in the patient, notwithstanding that the patient can still be afflicted with the underlying disease. The term “treat”, in all its verb forms, is used herein to mean to relieve, alleviate, prevent, and/or manage at least one symptom of a disease in a subject. As used herein, an “effective amount” refers to an amount sufficient to elicit the desired biological response.

As used herein, “administration” of a disclosed composition encompasses the delivery to a subject a composition of the present invention, as described herein, or a prodrug or other pharmaceutically acceptable derivative thereof, using any suitable formulation or route of administration, e.g., as described herein.

The precise amount of the composition administered to a subject will depend on the mode of administration, the type and severity of COVID-19 and on the characteristics of the subject, such as general health, age, sex, body weight and tolerance to the composition. The skilled artisan will be able to determine appropriate dosages depending on these and other factors. Suitable dosages are known for approved agents and can be adjusted by the skilled artisan according to the condition of the subject, and the severity of COVID-19. In cases where no amount is expressly noted, an effective amount should be assumed.

The term “reduce” or other forms of the word, such as “reducing” or “reduction,” generally refers to the lowering of an event or characteristic (e.g., one or more symptoms, or the binding of one protein to another). It is understood that this is typically in relation to some standard or expected value, in other words it is relative, but that it is not always necessary for the standard or relative value to be referred to. In some embodiments, the term “reducing,” is used in the context of “reducing COVID-19 symptoms.

The term COVID-19 stands for coronavirus disease 2019 that is caused by infection with severe acute respiratory syndrome coronavirus 2 or SARS-CoV-2, and includes all variants derived from the original isolated virus.

The term “susceptibility” refers herein to the risk of SARS-CoV-2 infection from the presence of ACE2 on the immune cell surface. The term “susceptibility” also extends to the risk of SARS-CoV-2 infection by ACE2 cell surface expression on multiple immune cells and the risk this presents when circulating immune cells with productive SARS-Cov-2 replication spread the virus systemically, increasing the risk of severe COVID-19. The term “susceptibility” also pertains to the risk of long COVID-19, wherein a subject continues to experience symptoms of COVID-19 for an extended period of time.

The term “bolus” refers to a single dose administered at one time point.

The term “prognosis” refers to the likely course or forecast of the disease.

The term “immunosuppressive phenotype” refers to a cell marker that indicates the immune cell function is inhibited.

The term “proinflammatory phenotype” refers to a cell marker that indicates that the immune cell is functioning at a highly active state, which if chronic could be detrimental to the subject.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent or patent application was specifically and individually indicated to be incorporated by reference in its entirety. And, all patent applications, patents, and printed publications cited herein are incorporated herein by reference in the entireties, except for any definitions, subject matter disclaimers, or disavowals, and except to the extent that the incorporated material is inconsistent with the express disclosure herein, in which case the language in this disclosure controls.

SARS-CoV-2 viral components (RNA, proteins) have been identified in multiple organs including lungs, heart, intestines, brain, and kidneys, and in various body fluids such as mucus, saliva, urine, and cerebrospinal fluid, suggesting systemic infection of SARS-CoV-2 in COVID-19 patients. However, it remains unclear whether circulating immune cells can be infected by SARS-CoV-2 and contribute to the SARS-CoV-2 systemic distribution.

The SARS-CoV-2 virus binds to the human angiotensin-converting enzyme 2 (ACE2) receptor and uses a spike (S) protein for attachment and entry into cells.7 Expression of ACE2 has been described in several types of cells, such as hematopoietic stem cells, endothelial progenitor cells, alveolar epithelial cells, enterocytes of the small intestine, and arterial smooth muscle cells. To date, single-cell RNA sequencing studies from human tissues have identified no ACE2 mRNA expression in most tissue-resident macrophages and peripheral blood cells from healthy individuals however, immunostaining of post-mortem tissue from COVID-19 patients has revealed that lymph node CD169+ macrophages express ACE2 protein and contain SARS-CoV-2 nucleoprotein. These findings suggest that ACE2 protein expression in macrophages may be triggered by inflammatory signals.

Infection of human peripheral blood cells by SARS-CoV-2 has been debated because immune cells lack mRNA expression of both angiotensin-converting enzyme 2 (ACE2) and transmembrane serine protease type 2 (TMPRSS2). The present disclosure demonstrates that resting primary monocytes harbor abundant cytoplasmic ACE2 and TMPRSS2 protein and that circulating exosomes contain significant ace2 protein. Upon ex vivo tlr4/7/8 stimulation, cytoplasmic ACE2 was quickly translocated to the monocyte cell surface independently of ACE2 transcription, while™ PRSS2 surface translocation occurred in conjunction with elevated mRNA expression.

From emerging evidence, it has been inferred that SARS-CoV-2 transcripts are present in both tissue-resident and monocyte-derived alveolar macrophages in bronchoalveolar lavage samples from patients with severe COVID-19, and the virus appears to be able to actively replicate in infected alveolar macrophages. SARS-CoV-2 viral particles have also been found in CD163+ macrophages in lung tissue from COVID-19 patients. Given that SARS-CoV-2 viral RNA is present in the blood or plasma from a small percentage of COVID-19 patients (<15%), it appears hypothesized that SARS-CoV-2 may directly infect blood monocytes that upregulate ACE2 expression upon inflammatory stimulation, which may alter their anti-virus immune phenotype and allow SARS-CoV-2 to spread from peripheral circulation to organs and other tissues.

This disclosure describes how resting circulating blood cells harbor abundant cytoplasmic ACE2 protein with hardly detectable mRNA (FIGS. 1E and 1F) and cell surface expression, while circulating exosomes highly express ACE2 (FIG. 38). The disclosure further describes that with ex vivo stimulation with TLR7/8 ligand R848 and TLR4 ligand LPS, the cytoplasmic ACE2 quickly translocates to the cell surface in all myeloid cells independently of ACE2 transcription (FIGS. 28, 2C, 20, 2E, 2F, 2G, 2H, and 2I), while elevated TMPRSS2 mRNA (FIG. 1G) and surface expression are only present in monocytes/macrophages but not in other blood cells (FIG. 4A).

This disclosure describes how SARS-CoV-2 can infect blood monocytes/macrophages that co-express surface ACE2 and transmembrane serine protease 2 (TMPRSS2) after pre-stimulation with TLR4/7/8 ligands (FIG. 6A). This disclosure describes how the monocytes/macrophages co-expressing ACE2 and TMPRSS2 are efficiently infected by SARS-CoV-2, and this was completely blocked by the viral replication inhibitor Remdesivir (FIG. 6A). Additionally, TLR4/7/8-activated peripheral myeloid cells from patients with moderate to severe COVI0-19 produced less IFN[3, and produced more proinflammatory cytokines and had higher PO-L1 expression compared to healthy controls (FIG. 8A).

Notably, ACE2 surface translocation was positively associated with proinflammatory responses and PO-L1 expression in myeloid cells from COVI0-19 patients (FIG. 8F). Results described herein in the Examples demonstrate that TLR4/7/8-inducing endosomal ACE2 surface translocation and TMPRSS2 expression may be indispensable for SARS-CoV-2 infection of the circulating monocytes/macrophages that may serve as viral reservoirs for systemic dissemination, and surface ACE2 expression in myeloid cells may be an early event involved in SARS-CoV-2-induced severe proinflammation. This disclosure describes new mechanisms for the pathogenesis of SARS-CoV-2 infection and a potential path for its systemic infection.

This disclosure describes how the rapid translocation of ACE2 to the monocyte cell surface can be blocked by a compound that inhibits endosomal trafficking, endosidin 2 (ES2) (FIGS. 3C and 30). Thus, this disclosure describes a therapeutic strategy of targeting ACE2 membrane trafficking for preventing monocyte and macrophage infection, based on the finding that the rapid translocation of ACE2 to the cell surface was blocked by the endosomal trafficking inhibitor, ES2. Moreover, this disclosure shows how ACE2 enters monocytes and phagocytes by endocytosis/phagocytosis of exosomes and that blocking endosome recycling can block ACE2 translocation to the cell surface, and thus reduce the susceptibility to SARS-CoV-2 infection.

In some embodiments, the present disclosure describes a composition comprising ES2, or a pharmaceutically acceptable salt thereof, for use in inhibiting exocytosis in an immune cell. Inhibiting exocytosis in an immune cell can reduce ACE2 translocation to the cell membrane of an immune cell. In some aspects, the present disclosure describes a composition comprising ES2, or a pharmaceutically acceptable salt thereof, for use in inhibiting endocytosis recycling. Inhibiting endocytosis recycling in an immune cells can also reduce ACE2 translocation to the cell membrane of an immune cell. SAR-CoV-2 uses the ACE2 receptor to enter cells and reduced ACE2 expression at the immune cell surface can reduce SARS-CoV-2 infection susceptibility of the immune cell.

In other aspects, the present disclosure describes a composition comprising ES2 or a pharmaceutically acceptable salt thereof and a pharmaceutically acceptable excipient. In other aspects, the present disclosure provides a composition comprising ES2, wherein the composition is carried in a drug delivery particle. In some aspects, the present disclosure provides a drug delivery particle with an immune cell targeting moiety on the surface. The immune cell targeting moiety may target receptors on immune cells or may be a ligand known to be phagocytosed by immune cells. In some aspects, the present disclosure describes a drug delivery particle carrying a composition comprising ES2, wherein a moiety on the drug delivery particle targets and/or is phagocytosed by a macrophage or monocyte.

In some aspects, the present disclosure provides a method of reducing SARS-CoV-2 infection in a subject by administration of a therapeutically effective dosage of a composition comprising ES2, as disclosed herein, to a subject in need thereof. In some aspects, the subject is a human. In some aspects, the present disclosure provides methods for administering the composition comprising ES2 enterally or parenterally. In some aspects, the present disclosure provides methods of treating a subject, wherein the composition is administered by oral ingestion, inhalation, infusion (intravenous, subcutaneous, intracranial, epidural, or intramuscular), or combinations thereof.

In some aspects, the present disclosure describes methods for administering a composition comprising ES2 in a single bolus dosage. In some aspects, the present disclosure describes methods for administering the composition comprising ES2 in a repeated dosing regimen.

Some embodiments described herein are directed to a method for determining COVID-19 prognosis in a subject, which may include isolating PBMCs from the subject, and measuring the level of ACE2 cell surface expression by, for example, flow cytometry. For example, PBMCs can be isolated from a COVID-19 patient and the level of ACE2 on the surface of PBMCs can be determined by flow cytometry. The level of ACE2 on PBMCs in a healthy individual is nominal so an increased level would indicate increased susceptibility for SARS-CoV-2 infection in PBMCs. Since circulating monocytes and macrophage cells are receptive to productive SARS-CoV-2 replication this would indicate the increased risk of a more severe systemic infection. In some embodiments, the level of ACE2 on PBMCs is measured along with proinflammatory and immunosuppressive cell phenotypes.

In some aspects, the present disclosure describes a method for measuring proinflammatory and immunosuppressive cell phenotypes in addition to ACE2 by detecting cell surface markers informing on these phenotypes. The cell surface markers comprise PO-L1, CCL5, CD11 b, CD38, CD163, CD86, HLA-DR, or combinations thereof, and may be measured by flow cytometry.

Some embodiments described herein are directed to measuring ACE2 and proinflammatory and immunosuppressive cell phenotypes in specific immune cell types. In some aspects, the disclosure describes the isolation and sorting of PBMCS into BDCA3+ cDCs, CD16+ classic monocytes, CD68+ macrophages, or a combinations thereof by, for example, flow cytometry, and thereby measure ACE2 and proinflammatory and immunosuppressive cell phenotypes on specific immune cell types.

In some aspects, the disclosure describes a method wherein proinflammatory and immunosuppressive phenotypes are measured by detecting cell surface markers comprising PD-L1, CCL5, CD11 b, CD38, CD163, CD86, HLA-DR, or combinations thereof, by for example, flow cytometry. The proinflammatory and immunosuppressive phenotypes are not limited to the markers listed above, and may therefore include an extensive panel of proinflammatory and immunosuppressive molecules, including cytokines, chemokines, and said receptors, as described in Costela-Ruiz et al., SARS-CoV-2 infection: The role of cytokines in COVID-19 disease, Cytokine Growth Factor Rev. 2020 August; 54: 62-75, which is incorporated herein by reference.

In addition, the present disclosure describes a method wherein the level of ACE2, and proinflammatory and immunosuppressive phenotypes are measured at different time points to inform on the prognosis of a subject with COVID-19.

### Compositions and Formulations

In various embodiments, the present disclosure describes compositions and formulations containing the compound endosidin 2 (ES2), or a pharmaceutically acceptable salt thereof. ES2 has the structure:

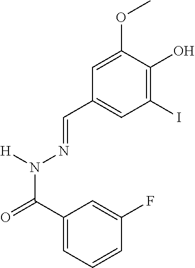 [SMILES: COc1cc(/C=N/NC(=O)c2cccc(F)c2)cc(I)c1O]

and is also known as 3-fluoro-benzoic acid, (2E)-2-[(4-hydroxy-3-iodo-5-methoxyphenyl)methylene]hydrazide. ES2 has a CAS Number 1839524-44-5, and PubChem Substance ID No: 329826011, and can be purchased commercially, for example from Cayman Chemical, Ann Arbor MI USA (Cat. No. 21888) or from Sigma-Aldrich, Inc., St. Louis, MO (Cat. No. SML1681). ES2 is a cell-permeable benzylidene-benzohydrazide that was initially shown to bind to the exocyst component of the 70 kDa (EX070) subunit of the exocyst complex (Kd=253 μM, EX070A1).1 See Zhang et al. (2016), ES22 targets conserved exocyst complex subunit EX070 to inhibit exocytosis; Proc. Natl. Acad. Sci. USA 113 E41, the disclosure of which is incorporated herein by reference in its entirety. ES2 binding inhibits exocytosis and endosomal recycling in plant and mammalian cells. ES2 disrupts protein trafficking between the endosome and plasma membrane, which enhances trafficking to the vacuole for degradation. It also inhibits recycling of endocytosed transferrin to the plasma membrane in Hela cells and can target multiple isoforms of mammalian EX070, resulting in misregulation of exocytosis.

Pharmaceutically acceptable carriers may contain inert ingredients which do not unduly inhibit the biological activity of the compositions. The pharmaceutically acceptable carriers should be biocompatible, e.g., non-toxic, non-inflammatory, non-immunogenic or devoid of other undesired reactions or side-effects upon administration to a subject. Standard pharmaceutical formulation techniques can be employed.

A pharmaceutically acceptable carrier, adjuvant, or vehicle, as used herein, includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and techniques for the preparation thereof. Except insofar as any conventional carrier mediums are incompatible with the compositions described herein, (such as by producing any undesirable biological effect or otherwise interacting in a deleterious manner with any other component(s) of the pharmaceutically acceptable composition), and their use is contemplated to be within the scope of this disclosure. As used herein, the phrase “side effects” encompasses unwanted or adverse effects of a therapy.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, etc. Preservatives and antioxidants can also be present in the composition, according to the judgment of the formulator.

In some embodiments, a composition of the present invention comprises a pharmaceutically acceptable salt. The term “pharmaceutically acceptable salts” is meant to include salts of the active compounds that are prepared with relatively nontoxic acids or bases, depending on the particular substituents found on the compounds described herein. If ES2 or a pharmaceutically acceptable salt, contains relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). ES2, or a pharmaceutically acceptable salt, may contain both basic and acidic functionalities that allow the compounds to be converted into either a base or acid salt.

Thus, ES2 may exist as a salt, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in a conventional manner. The parent form of ES2 may differ from the various salt forms in certain physical properties, such as solubility in polar solvents.

In addition to salt forms, the present invention may provide an ES2 compound in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention “Excipient” refers to any pharmacologically inactive, natural, or synthetic, component or substance that is formulated alongside (e.g., concomitantly), or subsequent to, the active ingredient of the present invention. In some embodiments, an excipient can be any additive, adjuvant, binder, bulking agent, carrier, coating, diluent, disintegrant, filler, glidant, lubricant, preservative, vehicle, or combination thereof, with which a extracellular vesicles of the present invention can be administered, and or which is useful in preparing a composition of the present invention.

In various embodiments, ES2 or a pharmaceutically acceptable salt thereof can be added to an excipient, carrier or diluent (herein referred to as “at least one excipient”) to make a pharmaceutically acceptable medicament for use in mammals, for example, humans. Excipients, include any such materials known in the art that are nontoxic and do not interact with other components of a composition. In other embodiments, an excipient can be used to confer an enhancement on the active ingredient in the final dosage form, such as facilitating absorption and/or solubility. In yet other embodiments, an excipient can be used to provide stability, or prevent contamination (e.g., microbial contamination). In other embodiments, an excipient can be used to confer a physical property to a composition (e.g., a composition that is a dry granular, or dry flowable powder physical form). Reference to an excipient includes both one and more than one such excipients. Suitable pharmaceutical excipients are described in Remington's Pharmaceutical Sciences, by E. W. Martin, the disclosure of which is incorporated herein by reference in its entirety.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. subjects to which administration of the pharmaceutical compositions is contemplated include, but are not limited to, non-human mammals, including agricultural animals such as cattle, horses, chickens and pigs, domestic animals such as cats, dogs, or research animals such as mice, rats, rabbits, dogs and non-human primates.

Relative amounts of the active ingredient, the pharmaceutically acceptable excipient or inert ingredient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient, ES2.

The composition comprising ES2 of the present disclosure may be formulated in any manner suitable for delivery. For example, the composition comprising ES2 may be carried in a drug delivery particle. The drug delivery particle may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

A targeting moiety on the drug delivery particle may target the particle to a receptor or marker expressed on the surface of a specific immune cell, such as a monocyte or macrophage. Strategies for targeting macrophage and monocytes, including a list of moieties, is described in Kelly et al., Targeted liposomal drug delivery to monocytes and macrophages, J Drug Deliv., Epub 2010 October 26, and are herein incorporated by reference. The drug delivery particle may be covered with ligands that are taken up by macrophage and monocytes. Possible ligands are listed in table 1 of the publication by Lameijer et al., titled, “Monocytes and macrophages as nonmedicinal targets for improved diagnosis and treatment of disease”, Expert Rev. Mol. Diagn. PMC 2014, July 25, and are herein incorporated by reference.

A pharmaceutical composition in accordance with the disclosure may be prepared, packaged, and/or sold in bulk, as a single unit dose form, and/or as a plurality of single unit doses. As used herein, a “unit dose” is discrete amount of the pharmaceutical composition comprising a predetermined amount of the active ingredient. The term “unit dosage form” refers to physically discrete units suitable as unitary dosage for subjects undergoing treatment, with each unit containing a predetermined quantity of ES2 calculated to produce the desired therapeutic effect, optionally in association with a suitable pharmaceutical carrier. The amount of the active ingredient is generally equal to the dosage of the active ingredient which would be administered to a subject and/or a convenient fraction of such a dosage such as, for example, one-half or one-third of such a dosage.

Formulations for delivery by a particular method (e.g., solutions, buffers, and preservatives, as well as droplet or particle size for intranasal administration) can be optimized by routine, conventional methods that are well-known in the art. For compositions that are in the form of aerosol formulations to be administered via inhalation, the aerosol formulations can be placed into pressurized acceptable propellants, such as dichlorodifluoromethane, propane, nitrogen or the like.

### Methods of Treatment

In various embodiments, the present disclosure provides a composition for the treatment of SARS-CoV-2 infection (also known colloquially as COVID-19). The composition comprises ES2, or a pharmaceutically acceptable salt thereof, for use in inhibiting exocytosis in an immune cell, wherein inhibiting exocytosis in the cell reduces translocation of ACE2 to the cell membrane of an immune cell, which reduces SARS-CoV-2 infection susceptibility of the immune cell. It has been unexpectedly shown that ES2, or a pharmaceutically acceptable salt thereof, has therapeutic benefit in reducing SARS-CoV-2 infection in a subject, wherein the subject is administered a therapeutically effective dosage of the composition comprising ES2, or a pharmaceutically acceptable salt thereof.

### Dosing and Administration

In some embodiments, the compositions of the present invention may be administered to a subject in need thereof. The terms “administration” or “administering” refer to the act of providing an composition of the present invention, e.g., the ES2 compound or pharmaceutically acceptable salt thereof, to a subject in need of treatment thereof.

The ES2 composition can be administered to humans and other animals orally, parenterally, intracisternally, intraperitoneally, buccally, as an oral or intranasal spray, such as an intranasal spray, a metered-dose inhaler, a nebulizer, a dry powder inhaler, or the like, depending on the severity of the infection being treated.

Therapeutically effective doses of the ES2 composition may be administered in concentrations ranging from about 0.01 mg/kg to about 1000 mg/kg and nested ranges within this broad range, e.g. 0.1 mg/kg to about 100 mg/kg. The ES2 concentration and dosage may be chosen based on the age and weight of the subject. The appropriate dosage and suitable age of the subject can be determined through clinical trials.

Efficacy of treatment or amelioration of covid-19 can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. in connection with the administration of the disclosed compositions, “effective against” SARS-CoV-2 infection, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating SARS-CoV-2 infection.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for the disclosed composition can also be judged using an experimental animal model for SARS-CoV-2 infection as known in the art. When using an experimental animal model, efficacy of treatment is evidenced when a statistically significant change is observed.

As claimed herein, a single bolus dose of the ES2 composition may be administered prophylactically after a subject tests positive for SARS-CoV-2 or after a subject begins to experience symptoms of the infection. As claimed herein, a repeated dosing regimen may include intermittent administration, wherein the composition is administrated for a period of time (which can be considered a “first period of administration”), followed by a time during which the composition is not taken or is taken at a lower maintenance dose (which can be considered “off-period”) followed by a period during which the composition is administered again (which can be considered a “second period of administration”). Generally, during the second phase of administration, the dosage level of the agent will match that administered during the first period of administration but can be increased or decreased as medically necessary. The intermittent dosing of the ES2 composition may be a single daily dose or one of multiple daily doses (e.g., about 1 to 4 or more times per day), weekly, or biweekly and may depend on the severity of disease, the dosing tolerance, and the therapeutic response to dosing. The unit dosage form can be the same or different for each dose.

In some embodiments, the ES2 composition may be administered enterally or parenterally. For example, an ES2 composition may be administered to a subject by oral ingestion, inhalation, infusion (intravenous, subcutaneous, intracranial, intraperitoneal, intrathecal, epidural, or intramuscular), or combinations thereof. Infusions may be administered by the implantation of a slow-release device, e.g., a mini-osmotic pump, to a subject. Other modes of delivery include, but are not limited to, the use of liposomal formulations or transdermal patches, etc.

### Oral Administration

Liquid dosage forms for oral administration include, but are not limited to, pharmaceutically acceptable emulsions, microemulsions, solutions, suspensions, syrups and elixirs. In addition to the ES2, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the composition is mixed with at least one inert pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, a dosage form may also comprise buffering agents.

A composition comprising ES2 may also be in microencapsulated form with one or more excipients as noted above. Solid dosage forms of tablets, dragees, capsules, pills, and granules can be prepared with coatings and shells such as enteric coatings, release controlling coatings and other coatings well known in the pharmaceutical formulating art. In such solid dosage forms, ES2 may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. Such dosage forms may optionally contain opacifying agents and may also be of a composition such that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

### Injected Formulations

In some embodiments, when treating a subject, an ES2 composition is administered by systemic intravenous (IV). Injectable preparations, for example, sterile injectable aqueous or oleaginous suspensions may be formulated according to known art using suitable dispersing or wetting agents and suspending agents. A sterile injectable preparation may also be a sterile injectable solution, suspension or emulsion in a nontoxic parenterally acceptable diluent or solvent, for example, a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid may be used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the ES2 composition, it is often desirable to slow absorption from subcutaneous or intramuscular injection. This may be accomplished by the use of a liquid suspension of crystalline or amorphous material with poor water solubility. The rate of absorption of the composition then depends upon its rate of dissolution that, in turn, may depend upon crystal size and crystalline form. Alternatively, delayed absorption of a parenterally administered composition form is accomplished by dissolving or suspending the composition in an oil vehicle. Injectable depot forms are made by forming microencapsule matrices of the composition in biodegradable polymers such as polylactide-polyglycolide. Depending upon the ratio of composition to polymer and the nature of the particular polymer employed, the rate of composition release can be controlled. Examples of other biodegradable polymers include poly(orthoesters) and poly(anhydrides). Depot injectable formulations are also prepared by entrapping the composition in liposomes or microemulsions that are compatible with body tissues.

### Pulmonary/Nasal Administration

An ES2 composition delivered by pulmonary/nasal administration can minimize systemic exposure. For pulmonary administration, preferably, an ES2 composition is delivered in a particle size effective for reaching the lower airways of the lung or sinuses. According to the invention, the composition can be delivered by any of a variety of inhalation or nasal devices known in the art for administration of a therapeutic agent by inhalation. The devices capable of depositing aerosolized formulations in the sinus cavity or alveoli of a patient include metered dose inhalers, nebulizers, dry powder generators, sprayers, and the like. Other devices suitable for directing the pulmonary or nasal administration of compositions are also known in the art. Many of such devices can use formulations suitable for the administration for the dispensing compositions as an aerosol. Such aerosols can be comprised of either solutions (both aqueous and non-aqueous) or solid particles.

In a metered dose inhaler (MDI), a propellant, the ES2 composition as disclosed herein, and any excipients or other additives are contained in a canister as a mixture including a liquefied compressed gas. Actuation of the metering valve releases the mixture as an aerosol, preferably containing particles in the size range of less than about 10 μm, in some embodiments, about 1 μm to about 5 μm, or from about 2 μm to about 3 μm. The desired aerosol particle size can be obtained by formulating a composition by various methods known to those of skill in the art, including jet-milling, spray drying, critical point condensation, or the like.

Preferred metered dose inhalers include those manufactured by 3M or Glaxo and employing a hydrofluorocarbon propellant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably, the propellant is a hydrofluorocarbon. The surfactant can be chosen to stabilize the composition as a suspension in the propellant, to protect the active agent against chemical degradation, and the like. Suitable surfactants include sorbitan trioleate, soya lecithin, oleic acid, or the like. In some cases, solution aerosols are preferred using solvents, such as ethanol. Additional agents known in the art for compound formulation can also be included in the formulation. One of ordinary skill in the art will recognize that the methods of the current invention can be achieved by pulmonary administration of the ES2 composition via devices not described herein.

Metered dose inhalers like the Ventolin@metered dose inhaler, typically use a propellant gas and require actuation during inspiration (See, e.g., WO 94/16970, WO 98/35888). Dry powder inhalers like Turbuhaler™ (Astra), Rotahaler@(Glaxo), Diskus@(Glaxo), Spiros™ inhaler (Dura), devices marketed by Inhale Therapeutics, and the Spinhaler@powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent@nebulizer (Mallinckrodt), and the Acorn 11@nebulizer (Marquest Medical Products) (U.S. Pat. No. 5,404,871 Aradigm, WO 97/22376), the above references are entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention.

In some embodiments, the ES2 composition is delivered by a dry powder inhaler or a sprayer. There are several desirable features of an inhalation device for administering the composition of the present invention. For example, delivery by the inhalation device is advantageously reliable, reproducible, and accurate. The inhalation device can optionally deliver small dry particles, e.g., less than about 10 μm, preferably about 1-5 μm, for good respirability.

A spray including the ES2 composition can be produced by forcing a suspension the composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

The inhalant formulation can include agents, such as an excipient, a buffer, an isotonicity agent, a preservative, a surfactant, and, preferably, zinc. The formulation can also include an excipient or agent for stabilization of the ES2 composition, such as a buffer, a reducing agent, a bulk protein, or a carbohydrate. Bulk proteins useful in formulating ES2 composition can include albumin, protamine, or the like. Typical carbohydrates useful in formulating inhaled compositions include sucrose, mannitol, lactose, trehalose, glucose, or the like. The ES2 composition can be formulated to include a surfactant, which can reduce or prevent surface-induced aggregation of the composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like.

The ES2 composition of the invention can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a composition solution through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of the composition either directly or through a coupling fluid, creating an aerosol including the ES2 composition.

Nebulizer formulations suitable for use with a nebulizer, either jet or ultrasonic can include agents, such as an excipient, a buffer, an isotonicity agent, a preservative, a surfactant, and, preferably, zinc. The formulation can also include an excipient or agent for stabilization of the composition, such as a buffer, a reducing agent, a bulk protein, or a carbohydrate. Bulk ES2 compositions may include albumin, protamine, or the like. Typical carbohydrates useful in formulating compositions include sucrose, mannitol, lactose, trehalose, glucose, or the like.

In some embodiments, the compositions of the present invention may be co-administered with one or more additional therapies. For example, the ES2 composition can be co-administered with another antiviral agent. By “co-administer” it is meant that a composition described herein is administered at the same time, just prior to, or just after the administration of additional therapies. The therapeutic drugs can be administered alone or can be co-administered to the patient. Co-administration is meant to include simultaneous or sequential administration of the components individually or in combination. Thus, the preparations can also be combined, when desired, with other active substances. As used herein, “sequential administration” includes that the administration of two agents (e.g., the agents described herein) do not occur on a same day.

As used herein, “concurrent administration” includes overlapping in duration at least in part. For example, when two compositions (e.g., any of the compositions described herein) are administered concurrently, their administration occurs within a certain desired time. The administration of the compositions may begin and end on the same day. The administration of one composition can also precede the administration of a second composition by day(s) as long as both compositions are taken on the same day at least once. Similarly, the administration of one composition can extend beyond the administration of a second composition as long as both agents are taken on the same day at least once. The composition do not have to be taken at the same time each day to include concurrent administration.

### EXAMPLES

The Examples in this specification are not intended to, and should not be used to, limit the invention; they are provided only to illustrate the invention.

Example 1: ACE2 is expressed on the cell surface of circulating immune cells upon ex vivo TLR stimulation. To determine whether human circulating immune cells express ACE2 protein under resting conditions, flow cytometry was used to examine the surface expression of ACE2 protein, and RNA extraction and quantitative real-time PCR (qRT-PCR) was used to measure mRNA expression in the principal immune cell populations, including T cells, B cells, CD14+ monocytes, classical dendritic cells (cDC), and plasmacytoid DCs (pDC) from fresh peripheral blood mononuclear cells (PBMC) from healthy donors (FIGS. 1A and 1B).

Methods. Blood samples were obtained from human subjects who provided written informed consent and samples were deidentified prior to processing under guidelines approved by the Henry Ford Health System Institutional Review Board. Donors with SARS-CoV-2 viral infection were enrolled in Detroit, MI, including subjects with moderate or severe disease following Centers for Disease Control and Prevention criteria as reported previously.42 In general, patients with moderate disease presented with cough, fever, myalgia, dyspnea, Sa02<94% on room air at rest or with exertion (walking); were shown to have pulmonary infiltrates by radiology imaging; and needed supplemental 02 therapy. Patients with severe disease had all above symptoms and had respiratory failure requiring mechanical ventilation. Identification of SARS-CoV-2 RNA from patient specimens was performed using PCR methods that were validated against the CDC reference method in Henry Ford's Microbiology Core Laboratory. Plasma from healthy donors (control) was measured for IgG antibodies to SARS-CoV-2 receptor binding domain Spike protein (RBD-S) using a Dxl 800 automated immunoassay analyzer (Beckman Coulter, Brea, CA). One sample from the healthy donors showed a positive result, indicating that one asymptomatic, previously infected individual was included in the healthy control group. All blood samples were collected in cell preparation tubes (CPT tubes, BO Biosciences, San Jose, CA) containing sodium heparin and were processed within 4 h of collection. All assays for mass cytometry used fresh blood cells, and the assays for flow cytometry used either fresh or frozen PBMCs.

Cell isolation and stimulation. For ex vivo stimulation assays analyzed by CyTOF, 250 μL of whole blood cells from CPT tubes were transferred to a 5 ml round-bottom tube with a lid (Thermo Fisher Scientific, Waltham, MA), followed by addition of RPMI medium (Sigma Aldrich, St. Louis, MO), resiquimod (R848, InvivoGen, San Diego, CA), or lipopolysaccharide (LPS, Sigma Aldrich, L4391) at a final concentration of 1 μg/ml. Brefeldin A (eBiosciences™, Thermo Fisher Scientific) and monensin (eBiosciences™, Thermo Fisher Scientific) were simultaneously added to the cells at a final concentration of 3 μg/ml and 2 μM, respectively. The cells were then incubated for 4 h at 37° C. with 5% C02. After stimulation, 50 μL of metal-conjugated surface antibody mixture was added to the tubes followed by incubation at 4° C. for 30 min. Next, 420 μL of PROT1 Proteomic Stabilizer (Smart Tube Inc., Las Vegas, NV) was added to the cells with incubation at room temperature (RT) for 10 min. The stained fixed cells were immediately placed at −80° C. for storage.

For ex vivo stimulation assays analyzed by flow cytometry, fresh PBMCs were isolated from CPT tubes using a standard protocol. Briefly, whole blood cells were diluted with Ca²+-free and Mg²+-free PBS buffer (Corning, NY) containing 2% fetal bovine serum (FBS, R&D Systems, Minneapolis, MN) and 2 mM EDTA (VWR Life Science, Radnor, PA) and slowly overlaid onto Ficoll-Paque™ (GE healthcare, Chicago, IL) at 2:1 ratio by volume. The samples were centrifuged at RT for 20 min at 800×g with no braking. Next, the mononuclear cell layer at the plasma-Ficoll interface was moved into a new 50 ml tube and washed with PBS buffer and red blood cell lysis buffer at RT for 5 min. The isolated PBMCs were used on the day of isolation or placed at −80° C. for storage. Two million fresh or thawed PBMCs were transferred to each well of a round-bottom 96-well culture plate (Thermo Fisher Scientific) in 200 μL of RPMI medium containing 10% FBS. The thawed PBMCs were recovered for 2 h at 37° C. with 5% C02. In some experiments, PBMCs were treated with different concentrations of ES2 (Sigma Aldrich) or 0.25% DMSO (the vehicle for ES2 at the highest concentration; ATCC, Manassas, VA) for 1-2 h during recovery stage. The fresh and recovered cells were then cultured with 1 μg/ml R848, LPS, or medium alone for 4 h. For TCR-independent T cell stimulation, PBMCs were cultured in RPMI medium containing 10% FBS in the presence of phorbol 12-myristate 13-acetate (PMA) (50 ng/ml) and ionomycin (1 μM) for a total of 4 h at 37° C. After incubation, the cells were washed with wash buffer in PBS containing 2% FBS and 1 mM EDTA prior to staining.

Flow cytometry methods. Single-cell suspensions were centrifuged at 450×g for 7 min, resuspended in ice-cold staining buffer (1×PBS with 2% FBS), and placed in a 96-well round-bottom plate (Thermo Fisher Scientific). After incubation with human Fe receptor blocking solution (Biolegend, San Diego, CA) at 4° C. for 15 min, cells were stained with a mixture of fluorescent surface antibodies at 4° C. for 30 min. For intracellular staining, the cells were fixed with IC Fixation Buffer (eBiosciences™, Thermo Fisher Scientific) at 4° C. for 30 min followed by incubation with fluorescent intracellular antibody cocktail in 1× Intracellular Fixation & Permeabilization Buffer (eBiosciences™, Thermo Fisher Scientific) at 4° C. for 30 min. The full list of fluorescent antibodies used is in Table 1 below. DAPI was added to the unfixed cells with surface staining at a final concentration of 1 μg/ml immediately before sample acquisition. The stained samples were then acquired on a FACSCelesta™ flow cytometer (BO Biosciences) using BO FACSDiva software version 8.0.2 (BO Biosciences). All data were analyzed using FlowJo 10.5.3 (BO Biosciences).

RNA extraction and quantitative real-time PCR. Total RNA was extracted with GenElute, purified with the Total RNA Purification Kit (Sigma Aldrich), and reverse-transcribed to cDNA with High Capacity cDNA Reverse Transcription Kits (Applied Biosystems, Foster City, CA). Quantitative real-time PCR (qRT-PCR) reactions were prepared using FastStart Universal SYBR Green Master (ROX, Roche) and carried out using QuantStudio 7 Flex Real-Time PCR System (Applied Biosystems). Data were collected using QuantStudio 7 Flex Real-Time PCR System software version 1.2 (Applied Biosystems) and analyzed using Microsoft Excel 2016 (Microsoft, Redmond, Washington). Quantitative gene expression data were normalized to GAPDH expression. The following primers were used: human ACE2: forward 5′-TGAAGTTGAAAAGGCCATCAG-3′ (SEQ ID NO: 1) and reverse 5′-GAGGTCCAAGTGTTGGCTGT-3′ (SEQ ID NO: 2); human TMPRSS2: forward 5′-GAGAAAGGGAAGACCTCAGAAG-3′ (SEQ ID NO: 3) and reverse 5′-GGTGTGATCAGGTTGTCATAGA-3′ (SEQ ID NO: 4); human GAPDH: forward 5′-CCTGCACCACCAACTGCTTA-3′ (SEQ ID NO: 5) and reverse 5′-GGCCATCCACAGTCTTCTGAG-3′(SEQ ID NO: 6); SARS-CoV-2 N1 nucleocapsid gene: forward 5′-GACCCCAAAATCAGCGAAAT-3′ (SEQ ID NO: 7) and reverse 5′-TCTGGTTACTGCCAGTTGAATCTG-3′ (SEQ ID NO: 8). SARS-CoV-2 sgRNA: forward 5′-GTAACAAACCAACCAACTTTCG-3′ (SEQ ID NO: 9) and reverse 5′-CATTGTTCACTGTACACTCGATC-3′ (SEQ ID NO: 10).

Results. Both circulating lymphoid and myeloid populations had negligible ACE2 protein on the cell surface, but ACE2 intracellular staining showed that the majority (>70%) of circulating immune cell populations, including T cells, B cells, monocytes, and DCs contained abundant ACE2 protein in the cytoplasm (FIGS. 1C and 1D). As expected, sorted CD14+ monocytes and CD3+ T cells from resting PBMC had nominal expression of ACE2 mRNA that was 10 to 100-fold lower than control cells (FIGS. 1E and 1F). TMPRSS2 mRNA is expressed in unstimulated T cells ex vivo (FIG. 1G).

TLR7/8 detects single-stranded RNA (such as the SARS-CoV-2 genome), while TLR4 can be activated directly by viral proteins, including SARS-CoV-2 spike proteins, or indirectly by danger signals triggered by viral infection. To determine whether ACE2 surface expression could be induced in PBMCs upon TLR stimulation, PBMCs were treated ex vivo with the TLR7/8 ligand resiquimod (R848) or the TLR4 ligand lipopolysaccharide (LPS, E. coli serotype 0111:B4) for 24 h.

Surface-localized ACE2 was markedly increased in CD14+ monocytes after treatment with either ligand, while marginal surface ACE2 was found in cDC and pDC following TLR stimulation (FIGS. 1H and 11). Robust surface ACE2 was observed in total T cells, CD4+ T cells, and CD8+ T cells after ex vivo stimulation with phorbol myristate acetate (PMA)/ionomycin (P/I) (FIGS. 3J and 3K). Collectively, circulating naïve immune cells have abundant intracellular ACE2 protein, and cell surface expression of ACE2 can be induced in monocytes and T cells upon activation.

TABLE 1
Antibodies used for flow cytometry and imaging cytometry

| Antibody | Clone | Conjugate | Company | Catalog# | Concentration |
|---|---|---|---|---|---|
| ACE2 | E-11 | Alexa Fluor 647 | Santa Cruz | sc-390851 | 0.2 μg/100 uL |
| CD3 | UCHT1 | FITC | BioLegend | 300406 | 1.5 μg/100 μL |
| CD3 | SK? | APC-CY7 | BO Biosciences | 557832 | 5 μL/100 μL |
| CD3 | OKT3 | SuperBright 780 | eBioscience | 78-0037-42 | 0.3 μg/100 μL |
| CD14 | M5E2 | PE | BioLegend | 301806 | 0.6 μg/100 μL |
| CD11c | Bu15 | Alexa Fluor 700 | BioLegend | 337220 | 3 μL/100 μL |
| CD19 | HIB19 | FITC | BioLegend | 302206 | 1.5 μg/100 μL |
| CD56 | NCAM16.2 | FITC | BO Biosciences | 340410 | 0.06 μg/100 μL |
| CD123 | 6H6 | PE-Dazzle 594 | BioLegend | 306034 | 3 μL/100 μL |
| Normal | | Alexa Fluor 647 | Santa Cruz | sc-24636 | 0.2 μg/100 μL |
| mouse IgG1 | | | | | |
| TMPRSS2 | H-4 | PE | Santa Cruz | sc-515727 | 0.2 μg/100 μL |

Example 2: Cytoplasmic ACE2 rapidly translocates to the cell surface upon TLR activation independent of ACE2 transcription. The mechanism by which surface expression of ACE2 occurs was examined by immune cell stimulation.

Methods. ACE2 protein levels were measured by immunoblot in monocytes purified from PBMC with or without R848 treatment and found that the protein levels of ACE2 in monocytes and total PMBCs. Imaging flow cytometry was used to monitor protein translocation in blood coi4+ monocytes 1 h to 4 h after treatment with R848 or LPS. The level of ACE2 mRNA transcript was measured in monocytes stimulated with R848 or LPS by qRT-PCR.

Immunoblot. Total protein was isolated using radioimmunoprecipitation assay (RIPA) buffer (Thermo Fisher Scientific, Waltham, MA). Equal amounts of protein were separated on 12% sodium dodecyl sulfate polyacrylamide gels and electro-transferred onto nitrocellulose membrane (Bio-Rad) at 1OOV for 2.5 h at 4° C. The membrane was blocked with 5% BSA for 1 hr at RT and probed with primary antibodies against ACE2 (1:600, AF933, R & D; 1:1,000, 4355S, Cell Signaling) and TMPRSS2 (1:1,000, sc-515727, Santa Cruz). GAPDH (1:1,000, 3683S, Cell Signaling) was used as internal control. HRP-conjugated rabbit anti-goat (1:2,000, 1721034, Bio-Rad) or goat anti-mouse (1:2,000, STAR117, Bio-Rad) were used as secondary antibodies. For immunoblotting of exosomal proteins, rabbit polyclonal CD63 antibody (1:1,000, Abeam) and goat-anti-rabbit (1:2,000, Cell Signaling) were used. Target proteins were visualized with an enhanced chemiluminescence detection system (GE Healthcare, NJ) using ChemiDoc™ MP imaging system and associated software (Bio-Rad, Hercules, CA).

Imaging flow cytometry. PBMCs were stained with anti-CD3/CD19/CD56-FITC, anti-CD14-PE, and anti-ACE2-Alexa Fluor 647 antibodies. DAPI (1 μg/ml) was used for nuclear imaging. Normal mouse IgG1-Alexa Fluor 647 was used as isotype control. In total, 200,000-400,000 events were collected for all samples on an ImageStream IS100 using 405 nm, 488 nm, and 642 nm laser excitation. Cell populations were hierarchically gated for single cells that were in focus, as described previously, and were positive for CD14 and negative for CD3, CD19, and CD56, which were defined as CD14+ monocytes. After the gates were applied, a total of 3,000-5,000 CD14+ monocytes were acquired for each sample and incorporated into the final analysis. Following data acquisition, the surface expression of ACE2 was measured by calculating the intensity feature ofACE2 signals using membrane mask in the IDEAS software package.

Immunofluorescence microscopy. PBMCs were blocked with human Fe Block, stained with ACE2-Alexa Fluor 647 (1:100) and CD14-PE (1:30) antibody, fixed with 4% PFA, permeabilized with Perm buffer, and stained with SARS-CoV-2 N-FITC antibody containing Fe Block. Tubes were sealed, the surface was decontaminated, and samples were transferred to a BSL2 lab. Cells were incubated with Hoechst 33342 (Invitrogen, 1:2,000) and centrifuged (180×g) into a 384-well plate (Perkin Elmer) in PBS for imaging. A Yokogawa Cell Voyager 8000 high content microscope was used for automated 4-color imaging. A 40×/1.0 NA water immersion objective was used with a 50 μm spinning disk confocal unit. The 405 nm/488 nm/561 nm/640 nm laser lines and corresponding emission filters (445/45 nm, 525/50 nm, 600/37 nm, 676/29 nm) were used to capture Hoechst-33342, FITC, PE, and Alexa Fluor-647, respectively. Maximum projection images were collected over a 10 μm range with a 0.3 μm step size. A total of 80 fields per well were collected and Ce11 Profiler46 was used to identify cells and then classify as CD14 positive by intensity measurements. Color images were produced in FIJ147, 48 and brightness/contrast was optimized per channel and was held constant for all cell images shown.

TLR stimulation. For confocal imaging analysis of ACE2 in MO, PBMCs were treated with 1 μg/ml R848 or medium alone for 24 h and stained with biotin-conjugated CD14 antibody followed by CD14+MO enrichment using Streptavidin Magnetic Beads (Thermo Scientific). The enriched MO reached over 85% purity and were immobilized onto slides using Cytospin™ 4 Cytocentrifuge (Thermo Scientific). The mounted cells were immediately fixed by IC Fixation Buffer at RT for 30 min, washed by PBS once, and blocked by human Fe block at RT for 30 min. The slides were then incubated with ACE2-Alexa Fluor 647 (1:50) antibody in PBS containing 3% BSA at 4° C. overnight. After 4 times of washes with PBS, the slides were incubated with Alexa Fluor 488-conjugated streptavidin (1:1,000) at RT for 1 h and then stained with DAPI (0.5 μg/ml) at RT for 2 min. Images were captured on an Olympus FV1000 confocal microscope using the 405 nm/473 nm/635 nm laser lines and corresponding emission wavelengths (461 nm, 520 nm, and 668 nm).

Results. The immunoblots showed that ACE2 protein levels in monocytes and PBMCs remained unaltered or slightly decreased (FIG. 2A), implying that the increase in ACE2 cell-surface expression observed in TLR-stimulated monocytes is independent of ACE2 gene transcription and that cytoplasmic ACE2 might translocate to the cell surface upon TLR stimulation. Consistent with results from conventional flow cytometry (FIGS. 1B, C), ACE2 was mainly located in the cytoplasm of resting CD14+ monocytes as seen by intracellular staining, while surface ACE2 signals were detected as early as 1 h after R848 or LPS stimulation, with gradual increases over the 2 to 4 h treatment (FIGS. 28, C, D, E, F, G).

Confocal fluorescence microscopy also confirmed the presence of intracellular ACE2 and CD14 in untreated monocytes and co-localization of ACE2 and CD14 on the monocyte cell surface after R848 treatment (FIG. 2H). These results suggest rapid translocation of cytoplasmic ACE2 to the monocyte surface upon TLR4/7/8 activation.

ACE2 mRNA expression remained comparable between sorted ACE2+ and ACE2− monocytes stimulated with R848 and was even lower in ACE2+ compared to ACE2-monocytes following LPS stimulation (FIG. 1D). Similarly, ACE2 mRNA remained comparable between sorted ACE2+ and ACE2− T cells following P/I stimulation (FIG. 1E). Example 3: Circulating exosomes contain ACE2 and cellular ACE2 translocation depends on endosomal trafficking. It is possible that ACE2 in mature immune cells may be carried over from early stages of immune cell development and differentiation. To determine the origin of cytoplasmic ACE2 protein in peripheral immune cells, the ACE2 mRNA was analyzed expression in 15 clusters identified from CD45+ hematopoietic cells in tissues from human embryos, including yolk sac-derived myeloid-biased progenitors, hematopoietic stem and progenitor cells, granulocyte-monocyte progenitors, lymphoid progenitors, monocytes, and macrophages.

Another source of ACE2 for immune cells could be extracellular vesicles. Exosomes as extracellular vesicles, have been recognized as a novel mode of intercellular communication and trafficking. Exosomes contain a large cargo of DNA, RNA, and proteins, which can be transferred to both neighboring and distant cells via circulation. ACE2 protein present in monocytes may be derived from ubiquitous ACE2-containing exosomes that have been released by ACE2-expressing cells, such as tissue epithelial cells and vascular endothelial cells.

Methods. Circulating exosomes (CD63+) were purified from the plasma of healthy donors to determine if the exosomes contained ACE2. Given that the putative destination of exosome-content delivery would be the endosomes, exosome-derived ACE2 may be in the endosomes of resting monocytes and could be translocated to the cell membrane through endosomal trafficking upon stimulation. To test this, PBMCs were pretreated with ES2 that binds to the exocyst complex subunit EX070 and inhibits endosomal recycling.

Exosome isolation and purification. Plasma exosomes were isolated and purified with ExoQuick ULTRA EV Isolation Kit (SBI System Biosciences, Palo Alto, CA), according to the manufacturer's protocol. Briefly, 67 μL of ExoQuick was added to 250 μL plasma after debris was removed and then incubated on ice for 30 min and centrifuged at 3,000×g for 10 min. The pellet was resuspended and loaded to a column for purification.

Results. The purified circulating exosomes (CD63+) from the plasma of healthy donors contained ACE2 protein (FIG. 3B). However, ACE2 mRNA was detected only in 3 cells of a macrophage subpopulation from a total of 1,231 cells (0.24%) among all clusters at the embryonic stage (FIG. 3A), suggesting that ACE2 protein storage in PBMCs or monocytes is unlikely from the early stage of hematopoietic stem and progenitor cell development. ACE2 translocated to the cell surface after treatment with R848 and LPS in a dose-dependent manner (FIG. 3C, D). Thus, ACE2 protein stored in co14+ monocytes likely derive from internalized circulating exosomes harboring ACE2 protein, which becomes deposited in the monocyte endosome at a steady state and quickly translocates to the cell membrane upon activation.

Example 4: TMPRSS2 is localized with ACE2 on the cell surface of monocytes upon TLR stimulation. SARS-CoV-2 viral entry requires not only binding to the ACE2 receptor, but also S protein priming by TMPRSS2, which cleaves the S protein and permits fusion of the viral and cellular membranes, or endocytosis and cleavage by cathepsin L. To determine the feasibility of SARS-CoV-2 viral entry, the presence of TMPR22 protein on the surface of monocytes and TMPR22 mRNA expression in monocytes was assessed. On the other hand, as surface ACE2 and TMPRSS2 were also detected (−5%) in untreated monocytes after 24 h culture. To determine whether, TMPRSS2 expression is induced by certain medium components, such as serum or PBMC-releasing factors, untreated PBMCs and PBMCs were treated with R848 or LPS in the presence or absence of serum for 24 hrs.

Methods. Flow cytometry and qRT-PCR were utilized as described in Example 1.

Results. Unexpectedly, TMPRSS2 was present in about 10%-40% of resting blood cells (FIGS. 4A, B). Interestingly, a marked increase in TMPRSS2 mRNA expression was observed in monocytes after R848 or LPS treatment (FIG. 4C), which was comparable in ACE2+ and ACE2− monocytes. Furthermore, TMPRSS2 protein was detected on the cell surface of S %-10% monocytes following 4 h treatment with R848 or LPS, and the frequency of TMPRSS2+ monocytes significantly increased (−20%-SO %) at 24 h and 48 h post-treatment compared to untreated groups (FIG. 40, E). Meanwhile, a robust increase in the frequency of ACE2+ TMPRSS2+ monocytes (−10%-1 S %) occurred after TLR stimulation for 24-48 h, suggesting that TLR stimulation induces localization of surface ACE2 and TMPRSS2 protein on monocytes.

Interestingly, although cell surface ACE2 localization was moderately induced (8.9%±0.98%, mean±SEM) in cDCs after R848 and LPS treatment, surface expression of TMPRSS2 was almost negligible (3.2%±0.87%, mean±SEM) upon stimulation (FIGS. SA, B). Very few ACE2+ TMPRSS2+ cDCs (1.1%±0.21%, mean±SEM) were detected after R848 or LPS stimulation. Furthermore, contrary to the robust appearance of surface ACE2 in T cells following P/I stimulation, surface TMPRSS2 was hardly detectable (<1%) in activated T cells (FIGS. SC, D). Thus, co14+ monocytes, not cDCs, mainly co-express surface ACE2 and TMPRSS2 following TLR stimulation, which could be utilized by SARS-CoV-2 for viral entry.

The frequency of untreated monocytes expressing surface ACE2 and/or TMPRSS2 remained detectable at comparable levels regardless of the presence of serum. However, the R848-treated and LPS-treated monocytes expressed relatively higher levels of surface ACE2 and TMPRSS2 in serum-containing medium than in serum-free medium (FIGS. S E, F). These results suggest that surface ACE2 and TMPRSS2 can be enhanced by certain serum nutrient factors when monocytes are activated. The factors driving surface localization of ACE2 and TMPRSS2 in the resting cells appear unrelated to serum nutrients and are possibly certain self-releasing factors produced by the cultured PBMCs.

Example 5: ACE2+co14+ monocytes are susceptible to infection with SARS-CoV-2 upon TLR activation. To determine the physiological significance and functionality of surface expression of ACE2 and TMPRSS2 upon TLR activation in monocytes, PBMCs were pretreated with R848, LPS, or medium alone for 2 h and infected cells with SARS-CoV-2.

Methods. To assess whether SARS-CoV-2 can actively replicate in the infected monocytes, PBMCs treated with remdesivir, an inhibitor of the viral RNA-dependent RNA polymerase that suppresses the rapid replication of a range of RNA viruses in human cells, including SARS-CoV-2. Recent evidence has shown that SARS-CoV-2 entry into lung cells can be significantly blocked by treatment with an anti-ACE2 antibody or camostat mesylate, an inhibitor of TMPRSS2. To determine whether co-expression of ACE2 and TMPRSS2 is required for SARS-CoV-2 infection of monocytes, PBMCs pretreated for 2 hrs with an anti-ACE2 antibody or an isotype control, or with camostat mesylate or its vehicle control, followed by incubation for 24 h with SARS-CoV-2 at MOI=3.

Virus infection. All SARS-CoV-2 infection-related work was performed in a Biosafety level 3 (BSL3) facility at the University of Michigan under the guidance of the Centers for Disease Control and Prevention. LNCaP, Vero E6, and Huh-7 cell lines were maintained in DMEM supplemented with 10% FBS, 100 U/ml penicillin, 100 U/ml streptomycin, and glutamine at 37° C. with 5% C02. All cell lines tested negative for mycoplasma. SARS-CoV-2 strain WA1/2020 (BEi resources, Catalog #NR-52281) was added in the BSL3 containment facility at a final working dilution equivalent to a multiplicity of infection (MOI) of 1 and allowed to incubate for 24 h at 37° C. Frozen PBMCs were thawed and seeded at 1×10⁶ cells/well on a 12-well cell culture plate. After resting at 37° C. with 5% C02 for 2 h, cells were cultured in the presence or absence of 1 μg/ml R848 (InvivoGen, Catalog #tlrl-r848, Dan Diego, CA), 1 μg/ml LPS (Sigma-Aldrich, Catalog #L4391), or 100 nM Remdesivir (MedChemExpress, Catalog #GS-5734-D5) for another 2 h at 37° C. After the second 2 h incubation, SARS-CoV-2 was then added to the cells at an MOI of 1 or 3 and allowed to incubate for 24 h at 37° C. in the presence of stimuli and/or inhibitor.

The infected Vero E6 and Huh-7 cells were treated with trypsin, blocked with human Fe Block, fixed with 4% paraformaldehyde (PFA), and resuspended in PBS. Tubes containing the fixed cells were sealed and decontaminated, and samples were transferred to a BSL2 lab for analysis. The infected PBMCs were blocked with PBS containing 3% BSA and human Fe receptor blocking solution and stained with fluorescent antibodies against surface ACE2, CD3, CD11c, CD123, CD14, and CD19 as listed in Supplementary information Table 3 and fixed with 4% PFA. Tubes containing fixed cells were sealed and decontaminated, and samples were transferred to a BSL2 lab. The purified SARS-CoV-2 nucleocapsid (N) antibody (Antibodies-online Inc., ABIN6952432, Limerick, PA) was labeled with the FITC conjugation kit-lightning-link (Abeam, ab102884). The fixed cells were then permeabilized and stained with the conjugated SARS-CoV-2 N-FITC antibody (1:400) in 1× Intracellular Fixation & Permeabilization Buffer (eBiosciences™) at 4° C. for 30 min prior to flow cytometry analysis.

Results. Infected cells were identified by the presence of cytoplasmic SARS-CoV-2 nucleocapsid (N) protein as measured by flow cytometry. As shown in FIG. 6A, a drastic increase in the frequency of ACE2+CD14+ monocytes was observed after SARS-CoV-2 was added to cultures in the absence of R848 or LPS stimulation, suggesting that ACE2 translocation could be triggered by viral infection alone. However, without stimulation, very few (<1%) ACE2+CD14+ monocytes were infected with the SARS-CoV-2, while the frequency of infected ACE2+CD14+ monocytes dramatically increased (up to −10%) after stimulation with R848 or LPS in a viral dose-dependent fashion (FIG. 6A). Interestingly, a significant proportion (up to −18%) of R848 and LPS-treated ACE2-co14+ monocytes were also infected. These results suggest that SARS-CoV-2 could infect blood monocytes through both ACE2-dependent and ACE2-independent mechanisms, which is partially consistent with a recent study. Few, if any, cDCs were infected by SARS-CoV-2, despite expressing ACE2 on their cell surface upon treatment with TLR4/7/8 ligands (FIG. 68). Treatment with remdesivir had minimal effects on surface ACE2 expression but almost completely blocked SARS-CoV-2 infection of TLR-stimulated ACE2+co14+ monocytes and ACE2-CD14+ monocytes (FIG. 6A).

Immunofluorescence staining revealed that punctate viral N protein was mainly located in the cytoplasm of the infected ACE2+co14+ monocytes after R848 stimulation (FIG. 6C). Meanwhile, SARS-CoV-2 N RNA and subgenomic RNA (sgRNA) were dramatically increased in R848-treated cells after infection for 48-72 h (FIGS. 60, E). These results suggest that SARS-CoV-2 can replicate in TLR-activated monocytes, which is different from abortive infection of SARS-CoV-2 in monocyte-derived macrophages and monocyte-derived DCs and is consistent with SARS-CoV-2 active replication in blood neutrophils.

Almost 60%-80% reduction in the frequency of ACE2+ CoV-2 N+co14+ monocytes were found after inhibition of ACE2 or TMPRSS2 compared to corresponding controls (FIG. 6F, G). These results suggest that co-expression of surface ACE2 and TMPRSS2 directly contributes to SARS-CoV-2 infection of CD14+ monocytes.

Example 6: ACE2 surface translocation correlates with hyperinflammation and PD-L1 expression in blood myeloid cells. To characterize the myeloid compartment-induced immune responses associated with COVID-19 severity, peripheral blood cells were stimulated from healthy control (HC) and patients with moderate or severe COVID-19 (see Table 2 for patient demographics) for 4 h ex vivo with R848 (FIG. 7A). The surface expression of ACE2 was further evaluated in blood myeloid cell subpopulations from COVID-19 patients and healthy controls before and after R848 ex vivo stimulation. To determine whether TLR4-mediated ACE2 surface translocation in myeloid populations is also associated with COVID-19 severity, peripheral blood cells were treated from the same patient cohorts ex vivo with LPS for 4 hrs (FIG. 7A).

Methods. Antibodies were used for functional analysis by cytometry by time-of-flight (CyTOF) to detect cell lineage markers, including BOCA1+ cDCs, BDCA3+ cDCs, co14+ classical monocytes, co14dimco15+ nonclassical monocytes, CD68+ macrophages, and pDCs (FIG. 78). Cells were TLR-stimulated as described in Example 2. Whole blood samples from cohort (n=7 healthy controls (HC); n=15 moderate COVID-19 patients; n=16 severe COVID-19 patients) were stimulated ex vivo with or without R848 for 4 h and then stained with 38 metal-conjugated antibodies for mass cytometry analysis. Heatmap of the frequencies of peripheral blood myeloid populations expressing indicated markers with and without R848 stimulation.

TABLE 2
Demographics of cross-sectional cohort

| Parameter | Healthy (n = 7) | | Moderate (n = 15) | Severe (n = 16) | | P-value |
|---|---|---|---|---|---|---|
| Mean age (yr) (SD), | 59.6 (12.0), 35-70 | | 55.9 (13.5), 36-85 | 60.1 (14.4), 24-84 | | 0.67 |
| range | | | | | | |
| No. (%) of women | 2 | (28.5) | 7 (46.7) | 3 | (18.8) | 0.25 |
| No. (%) of patients of | | | | | | |
| race | | | | | | |
| Black | 5 | (71.5) | 9 (60.0) | 10 | (62.5) | |
| White | 2 | (28.5) | 2 (13.3) | 4 | (25.0) | 0.65 |
| Other | 0 | (0) | 4 (26.7) | 2 | (12.5) | |
| Time since admit day | N.A. | | 4.8 (7.2), 0-25 | 19.7 (17.4), 1-46 | | 0.0051 |
| (SD), range | | | | | | |

Mass cytometry (CyTOF). The metal-conjugated antibodies used for CyTOF were purchased (Fluidigm or The Longwood Medical Area CyTOF core, Boston, MA) or conjugated in house using MaxPar X8 labeling kits according to the manufacturer's instructions (Fluidigm) (Table 3). The frozen stained blood samples were placed at RT for 30-50 min until fully thawed then incubated with 1× Thaw-Lyse buffer (Smart Tube Inc.) at RT for 10 min. The lysis steps were repeated a few times until the pellet turned white. If intracellular staining was needed, the cells were washed with Maxpar Cell Staining Buffer (Fluidigm), permeabilized with BO Perm II working solution (BO Biosciences) at RT for 10 min, and incubated with intracellular antibody cocktail containing 100 U/ml heparin solution (Sigma-Aldrich) at 4° C. for 45 min. The cells were then incubated with Maxpar Fix and Perm Buffer (Fluidigm) containing 125 nM Cell-ID Intercalator-Ir solution (Fluidigm) at 4° C. overnight or up to 48 h before sample acquisition. Groups of samples (10-16/day) were assessed by Helios mass cytometry (Fluidigm) in 16 independent experiments using a flow rate of 45 μl/min in the presence of EQ Calibration beads (Fluidigm) for normalization. An average of 360,000±13,600 cells (mean±SEM) from each sample were acquired and analyzed by Helios. Gating was performed on the Cytobank platform (Cytobank, Inc. Santa Clara, CA) and FlowJo 10.5.3 (BO Biosciences).

TABLE 3
Antibodies used for functional analysis by mass cytometry

| Antibody | Clone | Metal | Company | Catalog# | Concentration |
|---|---|---|---|---|---|
| C045 | HI30 | 89Y | Fluidigm | 30890038 | 1.0 μ∪300 μL |
| C033 | WM53 | 141Pr | Longwood* | N.A. | 0.5 μ∪300 μL |
| C019 | HI819 | 142Nd | Fluidigm | 31420018 | 0.5 μ∪300 μL |
| ACE2 | Polyclonal | 143Nd | R & O | AF933 | 0.5 μ∪300 μL |
| | Goat IgG | | | | |
| IFN- | IFNb/A1 | 144Nd | 8iolegend | 514002 | 0.125 μ∪300 μL |
| C0163 | GHI/61 | 145Nd | Fluidigm | 31450108 | 1.0 μ∪300 μL |
| TNF | MAb11 | 146Nd | Fluidigm | 31460108 | 0.125 μ∪300 μL |
| C0303 (80CA-2) | 201A | 147Sm | Fluidigm | 31470098 | 1.0 μ∪300 μL |
| C016 | 3G8 | 148Nd | Fluidigm | 31480048 | 0.5 μ∪300 μL |
| C01c (80CA1) | L161 | 149Sm | 8iolegend | 331502 | 0.25 μ∪300 μL |
| CCL4 (MIP-1b) | 021-1351 | 150Nd | Fluidigm | 31500048 | 0.125 μ∪300 μL |
| C0123 | 6H6 | 151Eu | Fluidigm | 31510018 | 0.5 μ∪300 μL |
| C066b | 80H3 | 152Sm | Fluidigm | 31520118 | 0.5 μ∪300 μL |
| CXCL10 (IP-10) | J03406 | 153Eu | 8iolegend | 519502 | 0.125 μ∪300 μL |
| IL-1b | H1b-27 | 154Sm | 8iolegend | 511601 | 0.125 μ∪300 μL |
| C036 | 5-271 | 155Gd | Fluidigm | 31550128 | 0.5 μ∪300 μL |
| IL-6 | MQ213A5 | 156Gd | Fluidigm | 31560118 | 0.125 μ∪300 μL |
| C027 | L128 | 158Gd | Fluidigm | 31580108 | 0.5 μ∪300 μL |
| C011c | 8u15 | 159Tb | Longwood | N.A. | 0.5 μ∪300 μL |
| IL-8 | 8H0814 | 160Gd | 8ioLegend | 514602 | 0.125 μ∪300 μL |
| C080 | 2010.4 | 1610y | Fluidigm | 31610238 | 0.5 μ∪300 μL |
| CCL3 | 93333R | 1620y | R & O | MA89849 | 0.125 μ∪300 μL |
| IL-12 (p70) | REA123 | 1630y | 8iolegend | 511002 | 0.125 μ∪300 μL |
| CCL5 (RANTES) | M80 | 1640y | 8iolegend | 515502 | 0.5 μ∪300 μL |
| C040 | 5C3 | 165Ho | Fluidigm | 31650058 | 1.0 μ∪300 μL |
| IL-10 | JES3907 | 166Er | Fluidigm | 31660088 | 0.5 μ∪300 μL |
| C038 | HIT2 | 167Er | Fluidigm | 31670018 | 0.5 μ∪300 μL |
| C0206 | 15-2 | 168Er | Fluidigm | 31680088 | 1.0 μ∪300 μL |
| PO-L1 | MIH1 | 169Tm | Fluidigm | 31690298 | 1.0 μ∪300 μL |
| HLA-OR | L243 | 170Er | Fluidigm | 31700138 | 0.5 μ∪300 μL |
| C068 | Y1/82A | 171Yb | Fluidigm | 31710118 | 1.0 μ∪300 μL |
| IgM | G10F5 | 172Yb | Fluidigm | 3172004B | 1.0 μ∪300 μL |
| CD141 (BDCA3) | LT27:295 | 173Yb | Fluidigm | 3173002B | 0.5 μ∪300 μL |
| CD86 | IT2.2 | 174Yb | Longwood | N.A. | 0.5 μ∪300 μL |
| CD14 | M5E2 | 175Lu | Fluidigm | 3175015B | 0.5 μ∪300 μL |
| IFNa | MHM-88 | 176Yb | BO | 551795 | 0.5 μ∪300 μL |
| | | | Biosciences | | |
| CD3 | UCHT1 | Qdot 605 | Life | 010054 | 1.0 μ∪300 μL |
| | | | Technologies | | |
| CD11b | ICRF44 | 209Bi | Fluidigm | 3209003B | 0.5 μ∪300 μL |

CyTOF data processing methods. All FCS files generated by CyTOF were normalized and concatenated, if necessary, using CyTOF Software version 6.7. All CyTOF processed files were also uploaded to the cloud-based Cytobank platform and beads, debris, doublets, and dead cells were manually removed by sequential gating shown in FIG. 3A. The CD45+CD66b− live singlets were either selected for ViSNE⁴⁴ analysis or gated manually with multiple cell lineage markers to define immune populations. The expression of checkpoint molecules and functional markers of each identified immune population was further analyzed by Cytobank platform or FlowJo software. The immune populations gated for each sample with less than 15 events were eliminated from the functional analysis. Heat maps and other plots were generated using Cytobank platform, GraphPad Prism 8.4.3 software (GraphPad, La Jolla, CA), or R 4.0.2 packages.

Results. Six myeloid cell populations were identified from peripheral blood based on the expression of lineage markers, including BOCA1+ cDCs, BDCA3+ cDCs, co14+ classical monocytes, CD14dimco15+ nonclassical monocytes, CD68+ macrophages, and pDCs (FIG. 7B) by CyTOF. Dynamic changes occurred in phenotypic and functional markers in the myeloid populations after R848 treatment (FIG. 7C). Without any treatment, most of the untreated myeloid cell populations from patients with severe COVID-19 disease expressed higher levels of inflammatory markers CCL5, CD11b, CD38 and CD163, and lower levels of HLA-DR and co-stimulatory CD86 than untreated cells from healthy controls (FIG. 70), consistent with results from another group. Proinflammatory cytokines IL-1[3, IL-6, and IL-8 were also highly expressed in untreated CD68+ macrophages from patients with severe disease, which is also consistent with previously published data.

After stimulating myeloid blood cells with R848, multiple myeloid subsets from COVID-19 patients had lower levels of IFNf3 and higher levels of TNF, IL-6, IL-12, CCL3, and CCL4 than cells from healthy controls (FIG. 7E). The R848-treated myeloid populations from COVID-19 patients also showed reduced expression of HLA-DR and enhanced expression of CD3S, CD6S, CDSO, and CD206, which was similar to the untreated condition. Interestingly, most myeloid populations from patients with severe COVID-19 expressed higher levels of PD-L1 than cells from moderately ill patients and control patients (FIG. 7F).

Surface expression of ACE2 was barely detected (O.S±0.1%; mean±SEM) in any of the untreated myeloid cells; however, RS4S treatment dramatically enhanced surface expression of ACE2 in myeloid subsets from COVID-19 patients and healthy controls, especially in CD14+ classic monocytes (11.0±2.7%) and CD6S+ macrophages (19.3±1.S %) (FIGS. SA, B), which was consistent with our flow cytometry data that showed increased surface ACE2 upon TLR7/S activation in CD14+ monocytes from healthy controls (FIGS. 1G, H). Compared to ACE2− cells, ACE2+ myeloid cells expressed lower levels of IL-10 and higher levels of IL-1!), CD11 b, and PD-L1, suggesting that ACE2 surface expression is positively associated with proinflammatory and immunosuppressive phenotypes (FIG. SC). Additionally, co-stimulatory molecules (CDSO and CDS6) and scavenger receptors (CD6S, CD163 and CD206) were also upregulated in ACE2-expressing cells, suggesting that ACE2+ myeloid cells may have reached a more advanced stage of maturation with enhanced phagocytic and migratory capacity. Intriguingly, upregulated surface expression of ACE2 was observed in the major myeloid populations from COVID-19 patients and healthy controls upon ex vivo LPS treatment (FIG. SD), which was coincident with the ACE2 phenotype in RS4S-treated myeloid cells (FIG. SE).

Importantly, surface expression of ACE2 in BDCA3+ cDCs, CD16+ monocytes, and pDCs from patients with severe disease was significantly higher than in cells from patients with moderate disease. Additionally, the majority of LPS-treated myeloid populations from patients with severe disease expressed higher levels of PD-L1 than cells from patients with moderate disease and control cells (FIG. SF), coinciding with results from RS4S treatment (FIG. 7F). These results collectively indicate that ACE2 cell surface translocation induced by TLR4/7/S activation positively correlates with proinflammatory responses and PD-L1 expression in the myeloid cell compartment from COVID-19 patients.

Statistics and Reproducibility for all Examples. No statistical method was used to predetermine sample sizes. All data were collected from at least two independent experiments. For categorical data, the two-tailed Fisher's exact test was used. For all continuous independent variables, if the data were not normally distributed as tested by the Shapiro-Wilk test, a nonparametric Mann-Whitney U test was used. For normally distributed data, if variances were equal, the Student's unpaired two-tailed t test was used; otherwise, the unpaired two-tailed t test with Welch's correction was used. Statistical analyses were performed with GraphPad Prism S.4.3 software or R 4.0.2 packages. Statistical significance is displayed as: *, P<0.05; **, P<0.01; ***, P<0.001.

1. A composition comprising the compound endosidin 2, or a pharmaceutically acceptable salt thereof, for use in inhibiting exocytosis in an immune cell, wherein inhibiting exocytosis in the cell reduces translocation of ACE2 to the cell membrane of an immune cell, which reduces SARS-CoV-2 infection susceptibility of the immune cell.

2. The composition according to claim 1, wherein the composition contains a pharmaceutically acceptable excipient.

3. The composition according to claim 1, wherein the composition is carried in a drug delivery particle.

4. The composition according to claim 3, wherein the surface of the drug delivery particle has a cell-targeting moiety.

5. The composition according to claim 4, wherein the cell-targeting moiety targets a receptor or marker expressed on the surface of an immune cell.

6. The composition according to claim 1, wherein the immune cell is a macrophage or a monocyte.

7. A composition comprising the compound endosidin 2, or a pharmaceutically acceptable salt thereof, for use in inhibiting endocytosis recycling, wherein inhibiting endocytosis recycling reduces translocation of ACE2 to the cell membrane of an immune cell, which reduces SARS-CoV-2 infection susceptibility of the immune cell.

8. The composition according to claim 7, wherein the composition contains a pharmaceutically acceptable excipient.

9. The composition according to claim 8, wherein the composition is carried in a drug delivery particle.

10. The composition according to claim 9, wherein the surface of the drug delivery particle has a cell-targeting moiety.

11. The composition according to claim 10, wherein the cell-targeting moiety targets a receptor or marker expressed on a monocyte or a macrophage.

12. A method of reducing SARS-CoV-2 infection in a subject, wherein the subject is administered a therapeutically effective dosage of the composition of claim 1.

13. The method according to claim 12, wherein the subject is human.

14. The method according to claim 12, wherein the composition is administered enterally or parenterally.

15. The method of claim 14, wherein the composition is administered by oral ingestion, inhalation, infusion (intravenous, subcutaneous, intracranial, epidural, or intramuscular), suppository, or combinations thereof.

16. The method of claim 12, wherein the composition is administered in a single bolus dose.

17. The method of claim 12, wherein the composition is administered in a repeated dosing regimen.

18. A method for determining COVID-19 prognosis in a subject, comprising isolating PBMCs from the subject, and measuring the level of ACE2 cell surface expression by flow cytometry.

19. The method according to claim 18, wherein the subject is human.

20. The method according to claim 18, wherein the level of ACE2 on PBMCs is measured along with proinflammatory and immunosuppressive cell phenotypes.

21. The method according to claim 20, wherein proinflammatory and immunosuppressive cell phenotypes are measured by detecting cell surface markers comprising PD-L1, CCLS, CD11b, CD38, CD163, CD86, HLA-DR, or combination thereof by flow cytometry.

22. The method according to claim 18, wherein BDCA3+ cDCs, CD16+ classic monocytes, CD68+ macrophages, or a combination thereof are sorted from the isolated PBMCs.

23. The method according to claim 22, wherein the cell surface expression of ACE2 is measured along with proinflammatory and immunosuppressive phenotypes in specific immune cell types.

24. The method according to claim 23, wherein proinflammatory and immunosuppressive phenotypes are measured by detecting cell surface markers comprising PD-L1, CCLS, CD11b, CD38, CD163, CD86, HLA-DR, or combination thereof by flow cytometry.

25. The method according to claim 18, wherein the level of ACE2, and proinflammatory and immunosuppressive phenotypes, are measured at different time points.
